(19) United States
(12) Patent Application Publication
(10) Pub. No.: US 2008/0306067 A1
(43) Pub. Date: Dec. 11, 2008

(54) Modulators of Chemokine Receptor Activity

(76) Inventors: Klaus Weigand, Vienna (AT); Thomas Ullrich, Vienna (AT); Kate Hoeganauer, Vienna (AT); Philipp Lehr, Vienna (AT); Rohan Beckwith, Cambridge, MA (US)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/091,102

(22) PCT Filed: Oct. 23, 2006
(86) PCT No.: PCT/EP06/67675

§ 371 Date: Aug. 8, 2008

(30) Foreign Application Priority Data
Oct. 25, 2005 (GB) 0521743.5

(51) Int. Cl. C07C 275/40; C07D 257/04; A61K 31/41; A61K 31/17; C07D 413/02; A61K 31/426; A61K 31/4245; A61P 37/08; A61P 11/00; C07D 271/10; C07D 277/02; A61K 31/5377; A61K 31/454; A61K 31/4439; C07D 401/02

(52) U.S. Cl. 514/236.2; 548/253; 514/381; 514/382; 514/340; 546/268.4; 564/50; 514/597; 514/326; 546/210; 544/132; 514/371; 548/196; 514/364; 548/143

(57) ABSTRACT

A compound of formula (I), wherein substituents are as given above, useful in the treatment of a disease mediated by the action of CCR3, in particular inflammatory or obstructive airway diseases.

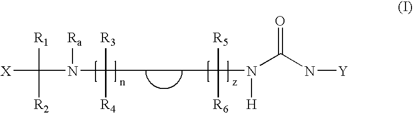

The present invention relates to modulators of chemokine receptor activity, e.g. the use of compounds of given formula in the preparation of a medicament.

In one aspect the present invention provides a compound of formula

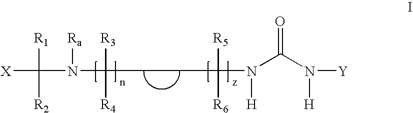

wherein
X is (C₆₋₁₈)aryl one or morefold substituted by halogen, cyano, hydroxy, carboxy, nitro, (C₁₋₈)alkyl, (C₁₋₈)alkoxy, (C₃₋₈)cycloalkyl, (C₃₋₈)cycloalkyl(C₁₋₄)alkoxy or (C₁₋₈)alkylamine, or unsubstituted or substituted (C₆₋₁₈)aryl annelated with (C₃₋₈)cycloalkyl,
R₁, R₂, R₃, R₄, R₅ and R₆ are independently selected from the group consisting of hydrogen, cyano, hydroxy, carboxy, nitro, (C₁₋₈)alkyl and (C₁₋₈)alkoxy,
Rₐ is (C₁₋₄)alkyl, (C₃₋₈)cycloalkyl,
n is zero or an integer from 1 to 8,
[symbol] is a single bond, a double bond, (C₃₋₈)cycloalkyl or absent,
z is an integer from 1 to 5,
Y is a cyclic group selected from the group consisting of (C₆₋₁₈)aryl and an optionally annelated 5 to 6 membered heterocyclic ring system, wherein at least one of the ring atoms is selected from N, O or S, said cyclic group being one or morefold substituted by halogen, (C₁₋₈)alkyl, (C₃₋₈)cycloalkyl, hydroxyl(C₁₋₄)alkyl, (C₁₋₆)alkoxycarbonyl, hydroxycarbonyl, (C₁₋₄)alkylcarbonylamino, (C₁₋₄)alkylaminocarbonyl, di(C₁₋₄)alkylaminocarbonyl, (C₆₋₁₈)arylaminocarbonyl, (C₆₋₁₈)aryl(C₁₋₂)alkylaminocarbonyl, (C₁₋₄)alkylamino(C₁₋₄)alkylaminocarbonyl, di(C₁₋₄)alkylamino(C₁₋₄)alkylaminocarbonyl, (C₃₋₆)cycloalkyl(C₁₋₄)alkylaminocarbonyl, (C₆₋₁₈)arylaminocarbonyl, (C₆₋₁₈)aryl(C₁₋₄)alkylaminocarbonyl,
   heterocyclyl(C₁₋₄)alkyl or heterocyclylcarbonyl, wherein heterocyclyl is a 5 to 6 membered heterocyclic ring system, wherein at least one of the ring atoms is selected from N, O or S,
   (C₆₋₁₈)aryl, or by an unsubstituted or substituted optionally annelated 5 to 6 membered heterocyclic ring system, wherein at least one of the ring atoms is selected from N, O or S.

In another aspect the present invention provides a compound of formula I, wherein
X is phenyl one or morefold substituted by halogen, cyano, hydroxy, carboxy, nitro, (C₁₋₄)alkyl, (C₁₋₄)alkoxy, (C₃₋₆)cycloalkyl, (C₃₋₆)cycloalkyl(C₁₋₂)alkoxy or (C₁₋₄)alkylamine, or
   unsubstituted or substituted phenyl annelated with (C₃₋₈)cycloalkyl,
R₁, R₂, R₃, R₄, R₅ and R₆ are independently selected from the group consisting of hydrogen, cyano, hydroxy, carboxy, nitro, (C₁₋₈)alkyl and (C₁₋₈)alkoxy,
Rₐ is (C₁₋₂)alkyl or (C₃₋₆)cycloalkyl,
n is zero or an integer from 1 to 4,
[symbol] is a double bond, (C₃₋₈)cycloalkyl or absent,
z is an integer from 1 to 4,
Y is phenyl one or morefold substituted by halogen, (C₁₋₈)alkyl, (C₃₋₆)cycloalkyl, hydroxy(C₁₋₄)alkyl, (C₁₋₆)alkoxycarbonyl, hydroxycarbonyl, (C₁₋₄)alkylcarbonylamino, (C₁₋₄)alkylaminocarbonyl, di(C₁₋₄)alkylaminocarbonyl, (C₆₋₁₈)arylaminocarbonyl, (C₆₋₁₈)aryl(C₁₋₂)alkylaminocarbonyl, (C₁₋₄)alkylamino(C₁₋₄)alkylaminocarbonyl, di(C₁₋₄)alkylamino(C₁₋₄)alkylaminocarbonyl, (C₃₋₆)cycloalkyl(C₁₋₄)alkylaminocarbonyl, (C₆₋₁₈)arylaminocarbonyl, (C₆₋₁₈)aryl(C₁₋₄)alkylaminocarbonyl,
   heterocyclyl(C₁₋₂)alkyl or heterocyclylcarbonyl, wherein heterocyclyl is a 5 to 6 membered heterocyclic ring system, wherein at least one of the ring atoms is selected from N, O or S, or by
   unsubstituted or substituted phenyl, tetrazolyl, tetrazolyl-methyl, benzothiazolyl, thiophenyl, pyrazolyl, furanyl, 2,3-dihydro-1H-indolyl, pyridinyl, 1,3,4-oxazolyl, 1H-imidazolyl, thiazolyl, or
   unsubstituted or substituted thiazolyl or pyridinyl.

In another aspect the present invention provides a compound of formula I, wherein
X is phenyl one to threefold substituted by fluoro, methyl, methoxy, hydroxy, cyclopropylmethoxy, or
   phenyl annelated with cyclopentyl,
R₁, R₂, R₃, R₄, R₅ and R₆ are independently selected from the group consisting of hydrogen, and hydroxy,
Rₐ is methyl or cyclopropyl,
n is zero or 1,
[symbol] is a double bond, cyclopropyl or absent,
z is an integer from 1 to 4,
Y is phenyl one or twofold substituted by bromo, chloro, methyl, ethyl, hydroxymethyl, hydroxycarbonyl, ethoxycarbonyl, t-butoxycarbonyl, methylcarbonylamino, methylaminocarbonyl, ethylaminocarbonyl, i-propylaminocarbonyl, dimethylaminocarbonyl, dimethylaminoethylaminocarbonyl, cyclopropylmethylaminocarbonylamino, phenylaminocarbonyl, benzylaminocarbonyl, pyridin-2-yl-methylaminocarbonyl, unsubstituted tetrazol-5-yl, 2H-tetrazol-5-yl-methyl, 1-methyl-1H-tetrazol-1-yl, 1-methyl-5H-tetrazol-1-yl, benzothiazol-2-yl substituted by 1-methyl-1H-tetrazol-1-yl, thiophen-2-yl, 1-benzyl-1H-pyrazol-4-yl, furan-2-yl, 2,3-dihydro-1H-indol-5-yl, pyridin-2-yl, pyridin-3-yl, pyridin-4-yl, tetrazol-5-yl-methyl, piperidine-1-carbonyl, morpholine-4-carbonyl, 4-methyl-piperazine-1-carbonyl, pyrrolidine-1-carbonyl, 1,3,4-oxadiazol-2-yl substituted by methyl, 1H-imidazol-2-yl one or twofold substituted by methyl, thiazol-2-yl substituted by methyl, methylaminocarbonyl or dimethylaminocarbonyl, pyridin-3-yl substituted by 2-methyl-tetrazol-1-yl, unsubstituted phenyl or phenyl substituted by methylaminocarbonyl, or
   thiazol-2-yl one or twofold substituted by methyl, methylcarbonyl, or dimethylaminocarbonyl, or
   pyridin-3-yl substituted by methyl-tetrazolyl.

In another aspect the present invention provides a compound of formula I, wherein

n is 1, z is 3, [symbol] is absent, R₃, R₄, R₅ and R₆ are hydrogen and X, Y, R₁ and R₂ are as defined above.

In another aspect the present invention provides a compound of formula I, wherein

n is 1, z is 3, [symbol] is absent, R₃ is hydroxyl, R₄, R₅ and R₆ are hydrogen and X, Y, R₁ and R₂ are as defined above.

In another aspect the present invention provides a compound of formula I, wherein
X is phenyl one or twofold substituted by fluoro or methyl,
R₁, R₂, R₃, R₄, R₅ and R₆ are independently selected from the group consisting of hydrogen, and hydroxy,
Rₐ is methyl,
n is zero or 1,
[symbol] is a double bond, cyclopropyl or absent,
z is an integer from 1 to 4,
Y is phenyl one or twofold substituted by methyl, ethyl, halogen, unsubstituted tetrazolyl, 1-methyl-1H-tetrazol-1-yl or 1-methyl-5H-tetrazol-1-yl, or benzothiazol-2-yl substituted by 1-methyl-1H-tetrazol-1-yl.

In a further aspect the present invention provides a compound of formula I, wherein
X is phenyl one- or twofold substituted by fluoro or methyl,
[symbol] is absent,
n is zero, z is 3 or 4,
Rₐ is methyl,
R₁, R₂, R₃, R₄, R₅, R₆ are hydrogen, and
Y is phenyl one- or twofold substituted by a substituent selected from the group consisting of (C₁₋₂)alkyl,
   bromo,
   unsubstituted or substituted 1H-tetrazol-5-yl, 2H-tetrazol-5-yl and tetrazol-1-yl;
X is phenyl onefold substituted by fluoro,
[symbol] is cyclopropyl,
m is 1, n is 1, z is 1,
Rₐ is methyl,
R₁, R₂, R₃, R₄, R₅, R₆ are hydrogen, and
Y is phenyl onefold substituted by 1H-tetrazol-5-yl;
X is phenyl onefold substituted by fluoro,
[symbol] is a double bond,
n is 1, z is 1,
Rₐ is methyl,
R₁, R₂, R₃, R₄, R₅, R₆ are hydrogen, and
Y is phenyl onefold substituted by 1H-tetrazol-5-yl;
X is phenyl onefold substituted by fluoro,
[symbol] is absent,
n is 1, z is 3,
Rₐ is methyl,
R₁, R₂, R₃, R₄ and R₅ are hydrogen,
R₆ are is hydroxyl, and
Y is phenyl onefold substituted by 1H-tetrazol-5-yl.

In another aspect the present invention provides a compound selected from the group consisting of
1-{4-[(4-Fluorobenzyl)-methylamino]butyl}-3-[3-(1-methyl-1H-tetrazol-5-yl)phenyl]-urea
1-[3-(1-Ethyl-1H-tetrazol-5-yl)phenyl]-3-{4-[(4-fluorobenzyl)methylamino]butyl}-urea
1-{4-[(4-fluorobenzyl)-methylamino]butyl}-3-[3-(1-isopropyl-1H-tetrazol-5-yl)phenyl]-urea
1-{4-[(4-fluorobenzyl)-methylamino]butyl}-3-[3-(1-propyl-1H-tetrazol-5-yl)phenyl]-urea
1-[3,5-Bis-(1-methyl-1H-tetrazol-5-yl)phenyl]-3-{4-[(4-fluorobenzyl)-methylamino]butyl}-urea
1-{3-[(4-Fluorobenzyl)-methylamino]propyl}-3-[3-(1-methyl-1H-tetrazol-5-yl)phenyl]-urea
1-{4-[(3,4-Dimethylbenzyl)-methylamino]butyl}-3-[3-(1-methyl-1H-tetrazol-5-yl)phenyl]-urea
1-{4-[(4-Fluorobenzyl)-methylamino]butyl}-3-[3-(1H-tetrazol-5-yl)phenyl]-urea
1-[3-(2-Ethyl-2H-tetrazol-5-yl)phenyl]-3-{4-[(4-fluorobenzyl)-methylamino]butyl}-urea
1-{4-[(4-Fluorobenzyl)-methylamino]butyl}-3-[3-(5-methyl-tetrazol-1-yl)phenyl]-urea
1-[3-Ethyl-5-(1-methyl-1H-tetrazol-5-yl)phenyl]-3-{4-[(4-fluorobenzyl)-methylamino]butyl}-urea
1-[3-Bromo-5-(1-methyl-1H-tetrazol-5-yl)phenyl]-3-{4-[(4-fluorobenzyl)-methylamino]butyl}-urea
1-{4-[(3,4-Dimethylbenzyl)-methylamino]butyl}-3-[3-ethyl-5-(1-methyl-1H-tetrazol-5-yl)phenyl]-urea
1-[3-Bromo-5-(1-methyl-1H-tetrazol-5-yl)phenyl]-3-{4-[(3,4-dimethylbenzyl)-methylamino]-butyl}-urea
1-(2-{[(4-Fluorobenzyl)-methylamino]methyl}-cyclopropylmethyl)-3-[3-(1-methyl-1H-tetrazol-5-yl)phenyl]-urea
1-{(E)-4-[(4-Fluorobenzyl)-methylamino]but-2-enyl}-3-[3-(1-methyl-1H-tetrazol-5-yl)phenyl)-urea
(±)-1-{4-[(4-Fluorobenzyl)-methylamino]-3-hydroxybutyl}-3-[3-(1-methyl-1H-tetrazol-5-yl)phenyl]-urea
1-{4-[(4-Fluorobenzyl)-methylamino]butyl}-3-[3-((1-methyl-1H-tetrazol-5-yl)-5-thiophen-2-yl)phenyl]urea
1-{4-[(4-Fluorobenzyl)-methylamino]-butyl}-3-[5-(1-methyl-1H-tetrazol-5-yl)-biphenyl-3-yl]-urea
1-[3-(1-Benzyl-1H-pyrazol-4-yl)-5-(1-methyl-1H-tetrazol-5-yl)-phenyl]-3-{4-[(4-fluorobenzyl)methylamino]-butyl}-urea
1-{4-[(4-Fluorobenzyl)-methylamino]-butyl}-3-[3-furan-2-yl-5-(1-methyl-1H-tetrazol-5-yl)-phenyl]-urea
1-[3-(2,3-Dihydro-1H-indol-5-yl)-5-(1-methyl-1H-tetrazol-5-yl)-phenyl]-3-{4-[(4-fluorobenzyl)methylamino]-butyl}-urea
N-[3′-(3-{4-[(3,4-Dimethylbenzyl)-methylamino]-butyl}-ureido)-5′-(1-methyl-1H-tetrazol-5-yl)-biphenyl-3-yl]-acetamide trifluoroacetate
N-[3′-(3-{4-[(4-Fluorobenzyl)-methylamino]-butyl}-ureido)-5′-(1-methyl-1H-tetrazol-5-yl)biphenyl-3-yl]-acetamide hydrochloride
1-{4-[(4-Fluorobenzyl)-methylamino]-butyl}-3-[3-(1-methyl-1H-tetrazol-5-yl)-5-pyridin-4-yl-phenyl]-urea
1-{4-[(4-Fluorobenzyl)-methylamino]-butyl}-3-[3-(1-methyl-1H-tetrazol-5-yl)-5-pyridin-3-yl-phenyl]-urea
3-(3-{4-[(3,4-Dimethylbenzyl)-methylamino]-butyl}-ureido)-N-methyl-5-(1-methyl-1H-tetrazol-5-yl)-benzamide
3-(3-{4-[(4-Fluorobenzyl)-methylamino]-butyl}-ureido)-N-methyl-5-(1-methyl-1H-tetrazol-5-yl)-benzamide
1-{4-[(3,4-Dimethylbenzyl)-methylamino]-butyl}-3-[3-tetrazol-5-yl-methyl)-phenyl]-urea trifluoroacetate
N-[4-bromo-3-(3-{4-[(4-fluorobenzyl)methylamino]butyl}ureido)phenyl]acetamide
N-[4-Chloro-3-(3-{4-[(4-fluorobenzyl)-methylamino]-butyl}-ureido)-phenyl]-acetamide
5-(3-{4-[(4-Fluorobenzyl)-methylamino]-butyl}-ureido)-N,N,N′,N′-tetramethyl-isophthalamide
5-(3-{4-[(3,4-Dimethylbenzyl)-methylamino]-butyl}-ureido)-N,N,N′,N′-tetramethyl-isophthalamide
1-{4-[N-(4-fluoro-3-methyl-benzyl)-N-methylamino]butyl}-3-[3-(1-methyl-1H-tetrazol-5-yl)phenyl]urea
1-{4-[(3-Fluoro-4-methyl-benzyl)-methylamino]-butyl}-3-[3-(1-methyl-1H-tetrazol-5-yl)-phenyl]-urea
1-{4-[(3,4-Difluorobenzyl)-methylamino]-butyl}-3-[3-(1-methyl-1H-tetrazol-5-yl)-phenyl]-urea
1-[3,5-Bis-(1-methyl-1H-tetrazol-5-yl)-phenyl]-3-{4-[(4-fluoro-3,5-dimethyl-benzyl)-methylamino]-butyl}-urea
1-[3,5-Bis-(1-methyl-1H-tetrazol-5-yl)-phenyl]-3-{4-[(4-fluoro-3-methyl-benzyl)-methylamino]-butyl}-urea
1-[3,5-Bis-(1-methyl-1H-tetrazol-5-yl)-phenyl]-3-{4-[(3-fluoro-4-methyl-benzyl)-methyl-amino]-butyl}-urea
1-[3,5-Bis-(1-methyl-1H-tetrazol-5-yl)-phenyl]-3-[4-(indan-5-yl-methyl-methylamino)-butyl]-urea
1-{4-[Cyclopropyl-(4-fluoro-benzyl)-amino]-butyl}-3-[3-(1-methyl-1H-tetrazol-5-yl)-phenyl]-urea
1-{4-[Cyclopropyl-(3,4-dimethylbenzyl)-amino]-butyl}-3-[3-(1-methyl-1H-tetrazol-5-yl)-phenyl]-urea
3-(3-{4-[(4-Fluorobenzyl)-methylamino]-butyl}-ureido)-N,N-dimethyl-5-(1-methyl-1H-tetrazol-5-yl)-benzamide
N-Ethyl-3-(3-{4-[(4-fluoro-benzyl)-methyl-amino]-butyl}-ureido)-5-(1-methyl-1H-tetrazol-5-yl)benzamide
3-(3-{4-[(4-Fluorobenzyl)-methylamino]-butyl}-ureido)-N-isopropyl-5-(1-methyl-1H-tetrazol-5-yl)benzamide
1-{4-[(4-Fluorobenzyl)-methylamino]-butyl}-3-[3-(1-methyl-1H-tetrazol-5-yl)-5-(piperidine-1-carbonyl)-phenyl]-urea
1-{4-[(4-Fluoro-benzyl)-methyl-amino]-butyl}-3-[3-(1-methyl-1H-tetrazol-5-yl)-5-(morpholine-4-carbonyl)-phenyl]-urea
1-{4-[(4-Fluorobenzyl)-methylamino]-butyl}-3-[3-(4-methyl-piperazine-1-carbonyl)-5-(1-methyl-1H-tetrazol-5-yl)-phenyl]-urea
1-{4-[(4-Fluorobenzyl)-methylamino]-butyl}-3-[3-(1-methyl-1H-tetrazol-5-yl)-5-(pyrrolidine-1-carbonyl)-phenyl]-urea
N-Cyclopropylmethyl-3-(3-{4-[(4-fluorobenzyl)-methylamino]-butyl}-ureido)-5-(1-methyl-1H-tetrazol-5-yl)-benzamide
3-(3-{4-[(4-Fluorobenzyl)-methylamino]-butyl}-ureido)-5-(1-methyl-1H-tetrazol-5-yl)-N-phenylbenzamide
3-(3-{4-[(4-Fluor-benzyl)-methylamino]-butyl}-ureido)-5-(1-methyl-1H-tetrazol-5-yl)-N-pyridin-2-ylmethyl-benzamide
N-Benzyl-3-(3-{4-[(4-fluorobenzyl)-methylamino]-butyl}-ureido)-5-(1-methyl-1H-tetrazol-5-yl)benzamide
N-(2-Dimethylaminoethyl)-3-(3-{4-[(4-fluorobenzyl)-methylamino]-butyl}-ureido)-5-(1-methyl-1H-tetrazol-5-yl)-benzamide
3-(3-{4-[(3,4-Dimethylbenzyl)-methylamino]-butyl}-ureido)-N,N-dimethyl-5-(1-methyl-1H-tetrazol-5-yl)-benzamide
N-Ethyl-3-(3-{4-[(3,4-Dimethylbenzyl)-methylamino]-butyl}-ureido)-5-(1-methyl-1H-tetrazol-5-yl)benzamide
3-(3-{4-[(3,4-Dimethylbenzyl)-methylamino]-butyl}-ureido)-N-isopropyl-5-(1-methyl-1H-tetrazol-5-yl)-benzamide
1-{4-[(3,4-Dimethylbenzyl)-methylamino]-butyl}-3-[3-(1-methyl-1H-tetrazol-5-yl)-5-(piperidine-1-carbonyl)-phenyl]-urea
1-{4-[(3,4-Dimethylbenzyl)-methylamino]-butyl}-3-[3-(1-methyl-1H-tetrazol-5-yl)-5-(morpholine-4-carbonyl)-phenyl]-urea
1-{4-[(3,4-Dimethylbenzyl)-methylamino]-butyl}-3-[3-(4-methyl-piperazine-1-carbonyl)-5-(1-methyl-1H-tetrazol-5-yl)-phenyl]-urea
1-{4-[(3,4-Dimethylbenzyl)-methylamino]-butyl}-3-[3-(1-methyl-1H-tetrazol-5-yl)-5-(pyrrolidine-1-carbonyl)-phenyl]-urea
N-Cyclopropylmethyl-3-(3-{4-[(3,4-dimethylbenzyl)-methylamino]-butyl}-ureido)-5-(1-methyl-1H-tetrazol-5-yl)-benzamide
3-(3-{4-[(3,4-Dimethylbenzyl)-methylamino]-butyl}-ureido)-5-(1-methyl-1H-tetrazol-5-yl)-N-phenyl-benzamide
N-Benzyl-3-(3-{4-[(3,4-dimethylbenzyl)-methylamino]-butyl}-ureido)-5-(1-methyl-1H-tetrazol-5-yl)-benzamide
3-(3-{4-[(3,4-Dimethylbenzyl)-methylamino]-butyl}-ureido)-5-(1-methyl-1H-tetrazol-5-yl)-N-pyridin-2-ylmethyl-benzamide
N-(2-Dimethylaminoethyl)-3-(3-{4-[(3,4-dimethylbenzyl)-methylamino]-butyl}-ureido)-5-(1-methyl-1H-tetrazol-5-yl)-benzamide
t-Butyl-3-(3-{4-[(4-Fluorobenzyl)-methylamino]-butyl}-ureido)-5-(1-methyl-1H-tetrazol-5-yl)benzoate
3-(3-{4-[(4-Fluorobenzyl)-methylamino]-butyl}-ureido)-5-(1-methyl-1H-tetrazol-5-yl)-benzoic acid hydrochloride
Ethyl-3-(3-{4-[(4-Fluorobenzyl)-methylamino]-butyl}-ureido)-5-(1-methyl-1H-tetrazol-5-yl)benzoate
3-{3-[4-(Indan-5-yl-methyl-methylamino)-butyl]-ureido}-N,N-dimethyl-5-(1-methyl-1H-tetrazol-5-yl)benzamide
3-(3-{4-[(4-Fluoro-3-methoxy-benzyl)-methylamino]-butyl}-ureido)-N,N-dimethyl-5-(1-methyl-1H-tetrazol-5-yl)-benzamide
3-(3-{4-[(4-Fluoro-3-hydroxy-benzyl)-methylamino]-butyl}-ureido)-N,N-dimethyl-5-(1-methyl-1H-tetrazol-5-yl)-benzamide
3-(3-{4-[(3-Cyclopropylmethoxy-4-fluorobenzyl)-methylamino]-butyl}-ureido)-N,N-dimethyl-5-(1-methyl-1H-tetrazol-5-yl)-benzamide
1-{4-[(4-Fluorobenzyl)-methylamino]-butyl}-3-[3-hydroxymethyl-5-(1-methyl-1H-tetrazol-5-yl)phenyl]-urea
1-{4-[(4-Fluorobenzyl)-methylamino]-butyl}-3-[3-methyl-5-(1-methyl-1H-tetrazol-5-yl)-phenyl]-urea
1-[3,5-Bis-(1-methyl-1H-tetrazol-5-yl)-phenyl]-3-{(E)-4-[(3,4-dimethyl-benzyl)-methyl-amino]-but-2-enyl}-urea
1-(5-Acetyl-4-methyl-thiazol-2-yl)-3-{4-[(4-fluorobenzyl)-methylamino]-butyl}-urea
1-(5-Acetyl-4-methyl-thiazol-2-yl)-3-{4-[(3,4-dimethylbenzyl)-methylamino]-butyl}-urea
1-{4-[(4-Fluorobenzyl)-methylamino]-butyl}-3-[5-(1-methyl-1H-tetrazol-5-yl)-pyridin-3-yl]-urea
2-(3-{4-[(4-Fluorobenzyl)-methylamino]-butyl}-ureido)-4-methyl-thiazole-5-carboxylic acid dimethylamide
2-(3-{4-[(3,4-Dimethylbenzyl)-methylamino]-butyl}-ureido)-4-methyl-thiazole-5-carboxylic acid dimethylamide
1-[3-(4,5-Dimethyl-1H-imidazol-2-yl)-phenyl]-3-{4-[(4-fluorobenzyl)-methylamino]-butyl}-urea
1-[3-(4,5-Dimethyl-1H-imidazol-2-yl)-5-(1-methyl-1H-tetrazol-5-yl)-phenyl]-3-{4-[(4-fluorobenzyl)methylamino]-butyl}-urea
1-{4-[(4-Fluorobenzyl)-methylamino]-butyl}-3-[3-(5-methyl-[1,3,4]oxadiazol-2-yl)-phenyl]-urea
1-{4-[(4-Fluorobenzyl)-methylamino]-butyl}-3-[3-(5-methyl-[1,3,4]oxadiazol-2-yl)-5-(1-methyl-1H-tetrazol-5-yl)-phenyl]-urea

If not otherwise defined herein
Alkyl includes (C₁₋₈)alkyl, e.g. (C₁₋₆)alkyl, such as e.g. (C₁₋₄)alkyl;
Cycloalkyl includes (C₃₋₈)cycloalkyl, such as e.g. cyclopropyl or cyclohexyl;
Alkoxy includes (C₁₋₈)alkoxy, e.g. (C₁₋₆)alkoxy, such as e.g. (C₁₋₄)alkoxy;
Aryl includes (C₆₋₁₈)aryl, e.g. phenyl; optionally anellated with (C₆₋₁₈)aryl, e.g. phenyl, with (C₃₋₈) cycloalkyl, e.g. cyclopentyl, or with heterocyclyl, e.g. heterocyclyl having 5 ring members and 1 N as heteroatom;
Heterocyclyl includes a 5 or 6 membered aromatic or non-aromatic ring having 1 to 4 heteroatoms selected from S, O and N; e.g. N, S; such as e.g. tetrazolyl, pyrazolyl, furanyl, pyridinyl, pyrolidinyl, piperidinyl, piperazinyl, morpholinyl, oxazolyl, oxadiazolyl, thiazolyl, thiophenyl,
optionally anellated with a further ring (system), e.g. anellated with one or more (C₆₋₁₈)aryl, e.g. phenyl, or anellated with heterocyclyl, such as e.g. indolyl, imidazolyl, benzothiazolyl;
Halogen includes fluoro, chloro, bromo;
Haloalkyl includes halo(C₁₋₄)alkyl, wherein halo is one or more halogen, preferably trifluoromethyl;

Any group may be unsubstituted or substituted, e.g. substituted by groups as conventional in organic chemistry, e.g. including groups selected from halogen, haloalkyl, alkylcarbonyloxy, alkoxy, hydroxy, amino, alkylcarbonylamino, aminoalkylcarbonylamino, hydroxyalkylamino, aminoalkylamino, alkylamino, dialkylamino, heterocyclyl having 5 or 6 ring members and 1 to 4 heteroatoms selected from N, O, S; (C₁₋₄)alkylheterocyclyl, wherein heterocyclyl having 5 or 6 ring members and 1 to 4 heteroatoms selected from N, O, S; hydroxy(C₁₋₄)alkylheterocyclyl, wherein heterocyclyl having 5 or 6 ring members and 1 to 4 heteroatoms selected from N, O, S; carboxyl, (C₁₋₄)alkylcarbonyloxy, amino(C₁₋₄)-alkylcarbonyloxy.

In a compound of formula I each single defined substitutent may be a preferred substituent, e.g. independently of each other substitutent defined.

Compounds used by or provided by the present invention are hereinafter designated as “compound(s) of (according to) the present invention”. A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes.

A salt of a compound of the present invention includes a metal salt or an acid addition salt. Metal salts include for example alkali or earth alkali salts; acid addition salts include salts of a compound of formula I with an acid, e.g. hydrogen fumaric acid, fumaric acid, naphthalin-1,5-sulphonic acid, hydrochloric acid, deuterochloric acid; preferably hydrochloric acid.

A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of pure isomers or mixtures thereof; e.g. optical isomers, diastereoisomers, cis/trans isomers. A compound of the present invention may e.g. contain asymmetric carbon atoms and may thus exist in the form of enantiomers or diastereoisomers and mixtures thereof, e.g. racemates. Any asymmetric carbon atom may be present in the (R)-, (S)- or (R,S)-configuration, preferably in the (R)- or (S)-configuration.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of formula I, where tautomers can exist.

In another aspect the present invention provides a process for the production of a compound of formula I comprising the steps

a. reacting a compound of formula

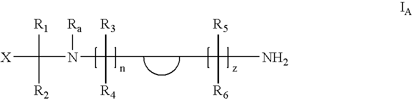

wherein X, Rₐ, R₁, R₂, R₃, R₄, R₅, R₆, n and z are as defined above,
   with
   b. a compound of formula

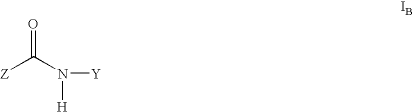

wherein Z is a protecting group/leaving group,
   to obtain a compound of formula I, and isolating a compound of formula I obtained from the reaction mixture.

In an intermediate of formula I_{A} or of formula I_{B} (starting materials), functional groups, if present, optionally may be in protected form or in the form of a salt, if a salt-forming group is present.

Protecting groups, optionally present, may be removed at an appropriate stage, e.g. according, e.g. analogously, to a method as conventional.

A compound of formula I thus obtained may be converted into another compound of formula I, e.g. or a compound of formula I obtained in free form may be converted into a salt of a compound of formula I and vice versa.

The above reaction may be carried out as appropriate, e.g. analogously to a method as conventional.

Intermediates (starting materials) of formula I_{A} or of formula I_{B} are known or may be prepared according, e.g. analogously, to a method as conventional or as described herein.

Any compound described herein, e.g. a compound of the present invention and intermediates of formula I_{A} or of formula I_{B} may be prepared as appropriate, e.g. according, e.g. analogously, to a method as conventional, e.g. or as specified herein.

The compounds of the present invention, e.g. including a compound of formula I, exhibit pharmacological activity and are therefore useful as pharmaceuticals. E.g., the compounds of formula I are useful in the preparation of a medicament for the treatment of a condition mediated by CCR3.

The compounds of the present invention act as CCR-3 receptor antagonists, thereby inhibiting the infiltration and activation of inflammatory cells, particularly eosinophils, and inhibiting allergic response. The inhibitory properties of the compounds of the present invention can be demonstrated in the following assay:

In this assay the effect of the compounds of the present invention on the binding of human eotaxin to human CCR-3 is determined. Recombinant cells expressing human CCR-3 are captured by wheatgerm agglutinin (WGA) polyvinyltoluidene (PVT) SPA beads (available from Amersham), through a specific interaction between the WGA and carbohydrate residues of glycoproteins on the surface of the cells. [¹²⁵I]-human eotaxin (available from Amersham) binds specifically to CCR-3 receptors bringing the [¹²⁵I]-human eotaxin in close proximity to the SPA beads. Emitted â-particles from the [¹²⁵I]-human eotaxin excite, by its proximity, the fluorophore in the beads and produce light. Free [¹²⁵I]-human eotaxin in solution is not in close proximity to the scintillant and hence does not produce light. The scintillation count is therefore a measure of the extent to which the test compound inhibits binding of the eotaxin to the CCR-3.

Preparation of Assay Buffer. 5.96 g HEPES and 7.0 g sodium chloride are dissolved in distilled H₂O and 1M aq. CaCl₂ (1 ml) and 1M aq. MgCl₂ (5 ml) are added. The pH is adjusted to 7.6 with NaOH and the solution made to a final volume of 1 L using distilled H₂O. 5 g of bovine serum albumin and 0.1 g NaN₃ are dissolved in the solution and the resulting buffer stored at 4° C. A Complete™ protease inhibitor cocktail tablet (available from Boehringer) is added per 50 ml of the buffer on the day of use.

Preparation of Homogenisation Buffer. Tris-base (2.42 g) is dissolved in distilled H₂O, the pH of the solution is adjusted to 7.6 with HCl and the solution obtained is diluted with distilled H₂O to a final volume of 1 l. The resulting buffer is stored at 4° C. A Complete™ protease inhibitor cocktail tablet is added per 50 ml of the buffer on the day of use.

Preparation of membranes: Confluent rat basophil leukaemia (RBL-2H3) cells stably expressing CCR3 are removed from tissue culture flasks using enzyme-free cell dissociation buffer and resuspended in phosphate-buffered saline. The cells are centrifuged (800 g, 5 minutes), the pellet obtained is resuspended in ice-cold homogenisation buffer using 1 ml homogenisation buffer per gram of cells and incubated on ice for 30 minutes. The cells are homogenised on ice with 10 strokes in a glass mortar and pestle. The homogenate is centrifuged (800 g, 5 minutes, 4° C.), the supernatant obtained is centrifuged (48,000 g, 30 minutes, 4° C.) and the pellet obtained is redissolved in Homogenisation Buffer containing 10% (v/v) glycerol. The protein content of the membrane preparation is estimated by the method of Bradford (Anal. Biochem. (1976) 72:248) and aliquots are snap frozen and stored at −80° C.

The assay is performed in a final volume of 250 μl per well of an Optiplate™ microplate (ex Canberra Packard). 50 μl of solutions of a test compound in Assay Buffer containing 5% DMSO (concentrations from 0.01 nM to 10 μM) are added to selected wells of the microplate. To determine total binding, 50 μl of the Assay Buffer containing 5% DMSO is added to other selected wells. To determine non-specific binding, 50 μl of 100 nM human eotaxin (ex R&D Systems) in Assay Buffer containing 5% DMSO is added to further selected wells. 50 μl of [¹²⁵I]-Human eotaxin (ex Amersham) in Assay Buffer containing 5% DMSO at a concentration of 250 μM (to give a final concentration of 50 μM per well), 50 μl of WGA-PVT SPA beads in Assay Buffer (to give a final concentration of 1.0 mg beads per well) and 100 μl of the membrane preparation at a concentration of 100 μg protein in Assay Buffer (to give a final concentration of 10 μg protein per well) are added to all wells. The plate is then incubated for 4 hours at room temperature. The plate is sealed using TopSeal-S™ sealing tape (ex Canberra Packard) according to the manufacturer's instructions. The resulting scintillations are counted using a Canberra Packard TopCount™ scintillation counter, each well being counted for 1 minute. The concentration of test compound at which 50% inhibition occurs (IC₅₀) is determined from concentration-inhibition curves in a conventional manner.

The compounds of the Examples herein below generally have IC₅₀ values below 1 μM in the above assay. For instance, the compound of example 1 has an IC₅₀ value of about 6 nM.

Most of the compounds of the Examples exhibit selectivity for inhibition of CCR-3 binding relative to inhibition of binding of the alpha-1 adrenergic receptor.

The inhibitory properties of the compounds of the present invention on binding of the alpha-1 adrenergic receptor can be determined in the following assay:

Cerebral cortices from male Sprague-Dawley rats (175-200 g) are dissected and homogenised in 10 volumes of ice cold 0.32 M sucrose (containing 1 mM MgCl₂ dihydrate and 1 mM K₂HPO₄) with a glass/Teflon homogeniser. The membranes are centrifuged at 1000×g for 15 minutes, the pellet discarded and the centrifugation repeated. The supernatants are pooled and centrifuged at 18,000×g for 15 minutes. The pellet is osmotically shocked in 10 volumes of H₂O and kept on ice for 30 minutes. The suspension is centrifuged at 39,000×g for 20 minutes, resuspended in Krebs-Henseleit buffer pH 7.4 (1.17 mM MgSO₄ anhydrous, 4.69 mM KCl, 0.7 mM K₂HPO₄ anhydrous, 0.11 M NaCl, 11 mM D-glucose and 25 mM NaHCO₃) containing 20 mM Tris, and kept for 2 days at −20° C. The membranes are thawed at 20-23° C., washed three times with Krebs-Henseleit buffer by centrifugation at 18,000×g for 15 minutes, left overnight at 4° C. and washed again 3 times. The final pellet is resuspended with a glass/Teflon homogeniser in 125 ml/100 membranes in the same buffer. A sample is taken to determine the protein concentration (using the Bradford Assay with gamma globulin as the standard) and the remainder aliquoted and stored at −80° C.

The resulting membranes are subjected to a radioligand binding assay. The assay is conducted in triplicate using 96 well plates containing [¹²⁵I]-HEAT (Amersham) (40 pM, K_{d}: 58.9±18.7 pM), unlabelled test compound and membrane (57.1 μg/ml) to yield a final volume of 250 μl (assay buffer containing 50 mM Tris-base and 0.9% (w/v) NaCl, pH 7.4). The plates are incubated at 37° C. for 60 minutes, after which rapid vacuum filtration over Whatman™ GF/C 96 well filter plates is carried out. Each plate is then washed three times with 10 ml of ice cold assay buffer using a Brandel Cell harvester (Gaithersburg, Md.). Following drying of the plates for 3 h. at 50° C., 40 μl of Microscint 20 is added to each well, the plates incubated at room temperature for a further 20 minutes and the retained radioactivity quantified in a Packard TopCount NXT™ scintillation counter.

Stock solutions of test compounds are dissolved initially in 100% DMSO and diluted with assay buffer to the required concentrations to yield 1% (v/v) DMSO. The concentration of test compound at which 50% inhibition occurs (IC₅₀) is determined from concentration-inhibition curves in a conventional manner.

Having regard to their inhibition of binding of CCR-3, the compounds of the present invention are useful in the treatment of conditions mediated by CCR-3, particularly inflammatory or allergic conditions. Treatment in accordance with the present invention may be symptomatic or prophylactic.

Accordingly, compounds of the present invention are useful in the treatment of inflammatory or obstructive airways diseases, resulting, for example, in reduction of tissue damage, bronchial hyper-reactivity, remodelling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial or viral infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. cortico-steroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), acute/adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The present invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

Having regard to their anti-inflammatory activity, in particular in relation to inhibition of eosinophil activation, compounds of the present invention are also useful in the treatment of eosinophil related disorders, e.g. eosinophilia, in particular eosinophil related disorders of the airways (e.g. involving morbid eosinophilic infiltration of pulmonary tissues) including hyper-eosinophilia as it effects the airways and/or lungs as well as, for example, eosinophil-related disorders of the airways consequential or concomitant to Löffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

The compounds of the present invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

The compounds of the present invention may also be used for the treatment of other diseases or conditions, in particular diseases or conditions having an inflammatory component, for example, treatment of diseases and conditions of the eye such as conjunctivitis, keratoconjunctivitis sicca, and vernal conjunctivitis, diseases affecting the nose including allergic rhinitis, e.g. atrophic, chronic, or seasonal rhinitis, inflammatory conditions of the gastrointestinal tract, for example inflammatory bowel disease such as ulcerative colitis and Crohn's disease, diseases of the bone and joints including rheumatoid arthritis, psoriatic arthritis, ankylosing spondylitis and systemic sclerosis, and other diseases such as cyctic fibrosis, pulmonary hypertension, atherosclerosis, multiple sclerosis, diabetes (type I), myasthenia gravis, hyper IgE syndrome and acute and chronic allograft rejection, e.g. following transplantation of heart, kidney, liver, lung or bone marrow.

The effectiveness of a compound of the present invention in inhibiting inflammatory conditions, for example in inflammatory airways diseases, may be demonstrated in an animal model, e.g. a mouse or rat model, of airways inflammation or other inflammatory conditions, for example as described by Szarka et al, J. Immunol. Methods (1997) 202:49-57; Renzi et al, Am. Rev. Respir. Dis. (1993) 148:932-939; Tsuyuki et al., J. Clin. Invest. (1995) 96:2924-2931; and Cernadas et al (1999) Am. J. Respir. Cell Mol. Biol. 20:1-8.

The compounds of the present invention are also useful as co-therapeutic compounds for use in combination with other drug substances such as anti-inflammatory, bronchodilatory, antihistamine or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the present invention may be mixed with the other drug substance in a fixed pharmaceutical composition or it may be administered separately, before, simultaneously with or after the other drug substance.

Such anti-inflammatory drugs include steroids, in particular glucocorticosteroids such as budesonide, beclamethasone, fluticasone, ciclesonide or mometasone, or steroids described in WO 02/88167, WO 02/12266, WO 02/100879, WO 04/039827 or WO 02/00679, especially those of Examples 3, 11, 14, 17, 19, 26, 34, 37, 39, 51, 60, 67, 72, 73, 90, 99 and 101; LTB4 antagonists such as those described in U.S. Pat. No. 5,451,700, also LY293111, CGS025019C, CP-195543, SC-53228, BIIL 284, ONO 4057 and SB 209247; LTD4 antagonists such as montelukast and zafirlukast; Dopamine receptor agonists such as cabergoline, bromocriptine, ropinirole and 4-hydroxy-7-[2-[[2-[[3-(2-phenylethoxy)propyl]sulfonyl]ethyl]-amino]ethyl]-2(3H)-benzothiazolone and pharmaceutically acceptable salts thereof (the hydrochloride being Viozan®—AstraZeneca); PDE4 inhibitors such as cilomilast (Ariflo® GSK), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID(TM) CC-10004 (Celgene), VM554/UM565 (Vernalis), T-440 (Tanabe), KW-4490 (Kyowa Hakko Kogyo), WO 92/19594, WO 93/19749, WO 93/19750, WO 93/19751, WO 99/16766, WO 01/13953, WO 03/104204, WO 03/104205, WO 04/000814, WO 04/000839 and WO 04/005258, WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945 and WO 04/045607, WO 04/037805 as well as those described in WO 98/18796 and WO 03/39544; A2a agonists such as those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618, WO 04/046083; and A2b antagonists such as those described in WO 02/42298.

Such bronchodilatory drugs include anticholinergic or antimuscarinic agents, in particular ipratropium bromide, oxitropium bromide, tiotropium bromide, CHF 4226 (Chiesi) and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. No. 5,171,744, U.S. Pat. No. 3,714,357, U.S. Pat. No. 5,171,744, WO 03/33495 and WO 04/018422; and beta (β)-2-adrenoceptor agonists such as albuterol (salbutamol), metaproterenol, terbutaline, salmeterol, fenoterol, procaterol, and especially, formoterol and pharmaceutically acceptable salts thereof, and compounds (in free or salt or solvate form) of formula (I) of WO 00/75114, which document is incorporated herein by reference, preferably compounds of the Examples thereof, especially a compound of formula

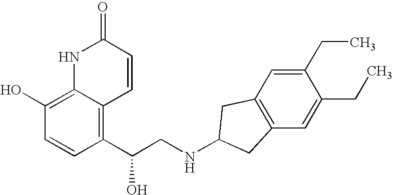

and pharmaceutically acceptable salts thereof, as well as compounds (in free or salt or solvate form) of formula (I) of WO 04/16601. Further suitable β-2-adrenoreceptor agonists include compounds such as those described in JP 05025045, US 2002/0055651, WO 93/18007, WO 99/64035, WO 01/42193, WO 01/83462, WO 02/066422, WO 02/070490, WO 02/076933, WO 03/24439, WO 03/72539, WO 03/42160, WO 03/91204, WO 03/42164, WO 03/99764, WO 04/11416, WO 04/16578, WO 04/22547, WO 04/32921, WO 04/33412, WO 04/37773, WO 04/37807, WO 04/39762, WO 04/39766, WO 04/45618 and WO 04/46083.

Such co-therapeutic antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in JP 2004107299, WO 03/99807 and WO 04/26841.

Combinations of compounds of the present invention and one or more steroids, beta-2 agonists, PDE4 inhibitors or LTD4 antagonists may be used, for example, in the treatment of COPD or, particularly, asthma. Combinations of compounds of the present invention and anticholinergic or antimuscarinic agents, PDE4 inhibitors, dopamine receptor agonists or LTB4 antagonists may be used, for example, in the treatment of asthma or, particularly, COPD.

Other useful combinations of compounds of the present invention with anti-inflammatory drugs are those with other antagonists of chemokine receptors, e.g. CCR-1, CCR-2, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770), CCR-5 antagonists described in U.S. Pat. No. 6,166,037 (particularly claims 18 and 19), WO 00/66558 (particularly claim 8), and WO 00/66559 (particularly claim 9), WO 04/018425 and WO 04/026873.

In accordance with the foregoing, the present invention also provides a method for the treatment of a condition mediated by CCR-3, for example an inflammatory or allergic condition, particularly an inflammatory or obstructive airways disease, which comprises administering to a subject, particularly a human subject, in need thereof an effective amount of a compound of formula (I) in a free or pharmaceutically acceptable salt form as hereinbefore described.

In another aspect the present invention provides the use of a compound of formula (I), in free or pharmaceutically acceptable salt form, as hereinbefore described for the manufacture of a medicament for the treatment of a condition mediated by CCR-3, e.g. an inflammatory or allergic condition, particularly an inflammatory or obstructive airways disease.

The compounds of the present invention may be administered by any appropriate route, e.g. orally, for example in the form of a tablet or capsule; parenterally, for example intravenously; by inhalation, for example in the treatment of inflammatory or obstructive airways disease; intranasally, for example in the treatment of allergic rhinitis; topically to the skin, e.g. in the treatment of atopic dermatitis; or rectally, e.g. in the treatment of inflammatory bowel disease.

In a further aspect, the present invention also provides a pharmaceutical composition comprising as active ingredient a compound of formula I in free or pharmaceutically acceptable salt form, optionally together with a pharmaceutically acceptable diluent or carrier therefor. The composition may contain a co-therapeutic agent such as an anti-inflammatory bronchodilatory or antihistamine drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g. patches. Compositions for inhalation may comprise aerosol or other atomizable formulations or dry powder formulations.

When the composition comprises an aerosol formulation, it preferably contains, for example, a hydro-fluoro-alkane (HFA) propellant such as HFA 134a or HFA227 or a mixture of these, and may contain one or more co-solvents known in the art such as ethanol (up to 20% by weight), and/or one or more surfactants such as oleic acid or sorbitan trioleate, and/or one or more bulking agents such as lactose. When the composition comprises a dry powder formulation, it preferably contains, for example, the compound of formula (I) having a particle diameter up to 10 microns, optionally together with a diluent or carrier, such as lactose, of the desired particle size distribution and a compound that helps to protect against product performance deterioration due to moisture e.g. magnesium stearate. When the composition comprises a nebulised formulation, it preferably contains, for example, the compound of formula (I) either dissolved, or suspended, in a vehicle containing H₂O, a co-solvent such as EtOH or propylene glycol and a stabiliser, which may be a surfactant.

The present invention includes (A) a compound of the present invention in inhalable form, e.g. in an aerosol or other atomisable composition or in inhalable particulate, e.g. micronised form, (B) an inhalable medicament comprising a compound of the present invention in inhalable form; (C) a pharmaceutical product comprising such a compound of the present invention in inhalable form in association with an inhalation device; and (D) an inhalation device containing a compound of the present invention in inhalable form.

Dosages of compounds of the present invention employed in practicing the present invention will of course vary depending, for example, on the particular condition to be treated, the effect desired and the mode of administration. In general, suitable daily dosages for administration by inhalation are of the order of 0.01 to 30 mg/kg while for oral administration suitable daily doses are of the order of 0.01 to 100 mg/kg.

The following abbreviations are used:

| | |
|---|---|
| ACN | acetonitrile |
| aq. | aq. |
| CH₂Cl₂ | methylene chloride |
| DMF | dimethylformamide |
| DMSO | dimethylsulfoxide |
| EtOAc | ethylacetate |
| EtOH | ethanol |
| MeOH | methanol |
| THF | tetrahydrofuran |
| TLC | thin layer chromatography |

### EXAMPLES

### Example 1

### 1-{4-[(4-Fluorobenzyl)-methylamino]butyl}-3-[3-(1-methyl-1H-tetrazol-5-yl)phenyl]-urea

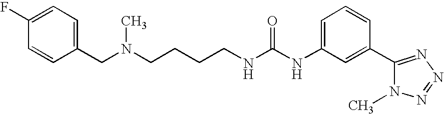

41 ml of methyl amine in THF are added to 7.05 g of 3-nitrobenzoyl chloride in 150 ml THF and stirred at ambient temperature for 2 hours. The reaction mixture obtained is diluted with EtOAc and washed 3 times with H₂O, brine, dried, filtered and concentrated. N-methyl-3-nitrobenzamide is obtained.

6.62 g of N-methyl-3-nitrobenzamide in 200 ml of ACN are added to 2.39 g of NaN₃ and the reaction mixture obtained is cooled to 0°. 10 g of trifluoromethanesulfonic anhydride are added drop-wise and the solution obtained is stirred at this temperature for 3.5 hours. The reaction mixture obtained is diluted with CH₂Cl₂ and poured into 1M NaOH. The organic layer obtained is separated, washed with H₂O, brine, dried, filtered and concentrated.

5-(3-nitrophenyl)-1-methyl-1H-tetrazole is obtained.

To 2.0 g of 5-(3-nitrophenyl)-1-methyl-1H-tetrazole in an EtOAc (25 ml) and MeOH (75 ml) solution, Pd/C (10%) is added under N₂. The reaction mixture obtained is flushed with N₂ and then with H₂. The hydrogenation reaction is conducted at 55 psi for 1 hour, the reaction mixture obtained is filtered through a plug of Celite and the filtrate obtained is concentrated. 3-(1-methyl-1H-tetrazol-5-yl) is obtained.

0.39 ml of phenyl chloroformate are added drop-wise to 539 mg of 3-(1-methyl-1H-tetrazol-5-yl)aniline) and 0.81 ml of 2,6-lutidine in 10 ml of CH₂Cl₂ and the reaction mixture obtained is stirred for 2 hours at ambient temperature. The reaction mixture obtained is poured into 1.0 M HCl and extracted with CH₂Cl₂. The combined organic layers obtained are washed with H₂O, brine, dried, filtered and concentrated.

Phenyl 3-(1-methyl-1H-tetrazol-5-yl)phenylcarbamate is obtained.

0.83 ml of Et₃N are added to 810 mg of N-1-(4-fluorobenzyl)-N-1-methylbutane-1,4-diamine. 2HCl and 925 mg of phenyl 3-(1-methyl-1H-tetrazol-5-yl)phenylcarbamate in 30 ml of ACN. The solution obtained is stirred at ambient temperature for 24 hours and concentrated.

1-{4-[(4-fluorobenzyl)methylamino]butyl}-3-[3-(1-methyl-1H-tetrazol-5-yl)phenyl]-urea is obtained.

MS ESI (M+H)⁺=412

The following compounds are prepared analogously as described in Example 1:

### Example 2

### 1-[3-(1-Ethyl-1H-tetrazol-5-yl)phenyl]-3-{4-[(4-fluorobenzyl)-methylamino]butyl}-urea

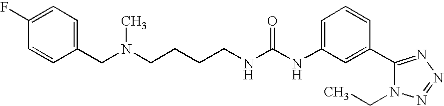

MS ESI (M+H)⁺=426

### Example 3

### 1-{4-[(4-fluorobenzyl)-methylamino]butyl}-3-[3-(1-isopropyl-1H-tetrazol-5-yl)phenyl]-urea

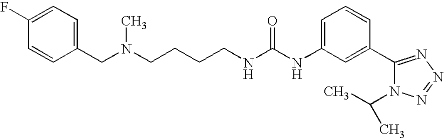

MS ESI (M+H)⁺=440

### Example 4

### 1-{4-[(4-fluorobenzyl)-methylamino]butyl}-3-[3-(1-propyl-1H-tetrazol-5-yl)phenyl]-urea

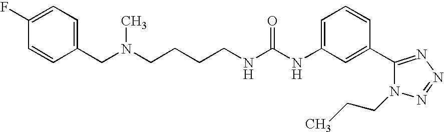

MS ESI (M+H)⁺=440

### Example 5

### 1-[3,5-Bis-(1-methyl-1H-tetrazol-5-yl)phenyl]-3-{4-[(4-fluorobenzyl)-methylamino]butyl}-urea

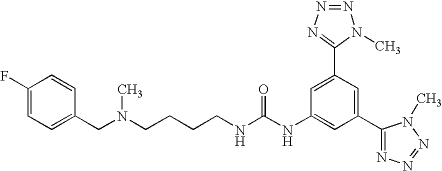

MS ESI (M+H)⁺=494

### Example 6

### 1-{3-[(4-Fluorobenzyl)-methylamino]propyl}-3-[3-(1-methyl-1H-tetrazol-5-yl)phenyl]-urea

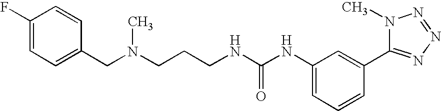

MS ESI (M+H)⁺=398

### Example 7

### 1-{4-[(3,4-Dimethylbenzyl)-methylamino]butyl}-3-[3-(1-methyl-1H-tetrazol-5-yl)phenyl]-urea

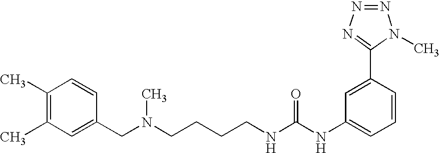

MS ESI (M+H)⁺=422

### Example 8

### 1-{4-[(4-Fluorobenzyl)-methylamino]butyl}-3-[3-(1H-tetrazol-5-yl)phenyl]-urea

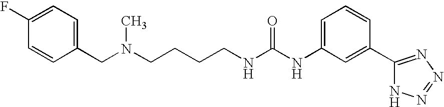

MS ESI (M+H)⁺=398

### Example 9

### 1-[3-(2-Ethyl-2H-tetrazol-5-yl)phenyl]-3-{4-[(4-fluorobenzyl)-methylamino]butyl}-urea

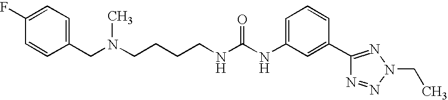

MS ESI (M+H)⁺=426

### Example 10

### 1-{4-[(4-Fluorobenzyl)-methylamino]butyl}-3-[3-(5-methyl-tetrazol-1-yl)phenyl]-urea

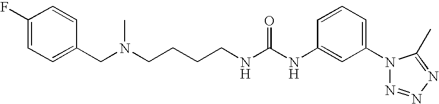

5 g Trifluoromethanesulfonic anhydride are added at 0° to a solution of 3.04 g of 3′-nitroanilide and 1.13 g of NaN₃ in 100 ml of ACN. The mixture obtained is stirred at this temperature for 3.5 hours.

The reaction mixture obtained is diluted with CH₂Cl₂ and poured into 1M NaOH. The organic layer obtained is isolated, dried, filtered and concentrated. 5-methyl-1-(3-nitrophenyl)-1H-tetrazole is obtained.

Pd/C (10%) is added under N₂ to 783 mg of 5-methyl-1-(3-nitrophenyl)-1H-tetrazole in a solution of 25 ml of EtOAc and 75 ml of MeOH. The reaction mixture obtained is flushed with nitrogen and then with hydrogen. The hydrogenation reaction is conducted at 55 psi for 1 hour, the reaction mixture obtained is filtered through a plug of Celite and the filtrate obtained is concentrated. 3-(5-methyl-tetrazol-1-yl)-aniline is obtained.

0.49 ml of phenyl chloroformate are added to 668 mg of 3-(5-methyl-tetrazol-1-yl)-aniline and 1.0 ml of 2,6-lutidine. The reaction mixture obtained is stirred for 1.5 hours at ambient temperature. The reaction mixture obtained is poured into 1.0 M HCl and extracted with CH₂Cl₂. The combined organic layers obtained are washed with H₂O, brine, dried, filtered and concentrated. Phenyl 3-(5-methyl-tetrazol-1-yl)-phenylcarbamate is obtained.

0.21 ml of Et₃N are added to 201 mg of N-1-(4-fluorobenzyl)-N-1-methylbutane-1,4-diamine. 2HCl and 210 mg of phenyl 3-(5-methyl-tetrazol-1-yl)-phenylcarbamate in 5 ml of ACN. The solution obtained is stirred at ambient temperature for 24 hours and concentrated.

1-{4-[(4-fluorobenzyl)methylamino]butyl}-3-[3-(5-methyl-tetrazol-1-yl)phenyl]-urea is obtained.

MS ESI (M+H)⁺=412

### Example 11

### 1-[3-Ethyl-5-(1-methyl-1H-tetrazol-5-yl)phenyl]-3-{4-[(4-fluorobenzyl)-methylamino]butyl}-urea

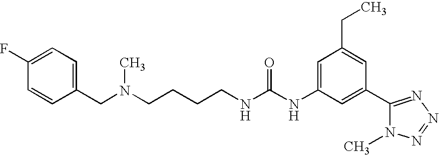

26.7 g of N-bromosuccinimide are added portion-wise to a stirred solution of 16.7 g of 3-nitrobenzoic acid in 50 ml of trifluoroacetic acid and 20 ml of sulfuric acid at 50°. The mixture obtained is stirred for 16 hours, cooled, poured into 200 ml of ice-H₂O and extracted 3 times with 100 ml of EtOAc. The combined organic layers obtained are dried, filtered, and concentrated, then triturated with CH₂Cl₂. 3-bromo-5-nitrobenzoic acid is obtained.

1 ml of N,N-dimethylformamide is added to a solution of 17.7 g of 3-bromo-5-nitrobenzoic acid and 12.55 ml of oxalyl chloride in 250 ml of CH₂Cl₂ and the mixture obtained is stirred at ambient temperature for 5 hours. The reaction mixture obtained is concentrated then re-concentrated twice with 100 ml of toluene to remove excess of oxalyl chloride. The residue obtained is dissolved in 100 ml of THF and added drop-wise to a solution of 108 ml of methylamine in THF at 0°. The mixture obtained is stirred at ambient temperature for 14 hours, filtered and the filtrate obtained is concentrated. 3-bromo-N-methyl-5-nitrobenzamide is obtained.

1.15 ml of NaN₃ are added to a suspension of 4.6 g of 3-bromo-N-methyl-5-nitrobenzamide in 100 ml of ACN at ambient temperature. The reaction mixture obtained is cooled to 0° prior to cautious addition of 5.5 g of triflic anhydride and stirred for 4 hours at this temperature. The solution obtained is quenched by addition of saturated aq. sodium hydrogen carbonate solution and extracted 3 times with 20 ml of EtOAc. The combined organic layers obtained are washed with sat. NaHCO₃, brine, dried, filtered and concentrated. 3-bromo-5-nitrophenyl)-1-methyl-1H-tetrazole is obtained.

188 mg of tetrakis(triphenylphosphine)palladium(0) are added to 460 mg of 3-bromo-5-nitrophenyl)-1-methyl-1H-tetrazole and 514 mg of tributylvinyltin in 5 ml of toluene. The mixture obtained is heated at reflux until complete by TLC (3 hours) and solvent is evaporated. 1-methyl-5-(3-nitro-5-vinylphenyl)-1H-tetrazole is obtained.

A catalytic amount of palladium on carbon (10%) is added to 200 mg of 1-methyl-5-(3-nitro-5-vinylphenyl)-1H-tetrazole in 3 ml of MeOH and the reaction mixture obtained is stirred under a H₂ atmosphere (1 atmosphere) for 2 hours. The reaction mixture obtained is filtered and the filtrate obtained is concentrated. Ethyl-5-(1-methyl-1H-tetrazol-5-yl)-aniline is obtained.

85 μl of 2,6-lutidine are added to 150 mg of ethyl-5-(1-methyl-1H-tetrazol-5-yl)-aniline in 4 ml of THF and the reaction mixture obtained is cooled to 0°. 97 μl of phenyl-chloroformate are added drop-wise and the solution obtained is stirred at this temperature for 1.5 hours. The mixture obtained is diluted with EtOAc and 0.1M HCl is added. The organic layer obtained is isolated and washed twice with 0.1M HCl, brine, dried, filtered and concentrated and trituration with toluene.

Phenyl-3-ethyl-5-(1-methyl-1H-tetrazol-5-yl)-phenylcarbamate is obtained.

37 μl of Et₃N are added to 30 mg of N-1-(4-fluorobenzyl)-N-1-methylbutane-1,4-diamine. 2HCl and 925 mg of phenyl 3-ethyl-5-(1-methyl-1H-tetrazol-5-yl)phenyl-carbamate in 1.0 ml of ACN. The reaction mixture obtained is stirred at ambient temperature for 18 hours and concentrated.

1-[3-Ethyl-5-(1-methyl-1H-tetrazol-5-yl)phenyl]-3-{4-[(4-fluorobenzyl)methylamino]butyl}-urea is obtained.

MS (M+H)⁺=441

Analogously as described for example 11 the following compounds are obtained:

### Example 12

### 1-[3-Bromo-5-(1-methyl-1H-tetrazol-5-yl)phenyl]-3-{4-[(4-fluorobenzyl)methylamino]butyl}-urea

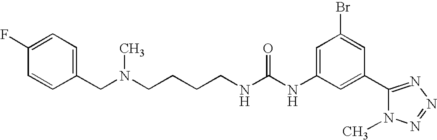

MS (M+H)⁺=492

### Example 13

### 1-{4-[(3,4-Dimethylbenzyl)-methylamino]butyl}-3-[3-ethyl-5-(1-methyl-1H-tetrazol-5-yl)phenyl]-urea

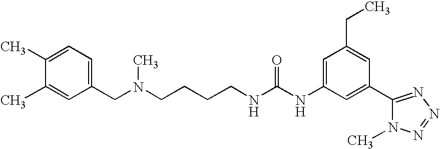

MS ESI (M+H)⁺=451

### Example 14

### 1-[3-Bromo-5-(1-methyl-1H-tetrazol-5-yl)phenyl]-3-{4-[(3,4-dimethylbenzyl)methylamino]butyl}-urea

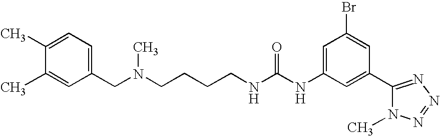

MS ESI (M+H)⁺=502

### Example 15

### 1-(2-{[(4-Fluorobenzyl)-methylamino]-methyl}-cyclopropylmethyl)-3-[3-(1-methyl-1H-tetrazol-5-yl)phenyl]-urea

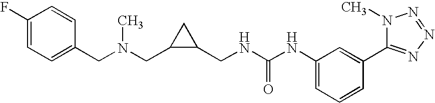

0.82 ml of 4-fluoro-N-methyl-benzylamine are added to a solution of 1.0 ml of ethyl 2-formyl-1-cyclopropane carboxylate in 24 ml acetic acid and acetonitrile (5:1) at ambient temperate. After stirring for 20 minutes, 0.48 g of sodium cyanoborohydride are added. The mixture obtained is stirred at ambient temperature for 2 hours. The reaction mixture obtained is quenched by addition of saturated aq. sodium hydrogen carbonate solution and extracted 3 times with 20 ml of EtOAc.

The combined organic layers obtained are washed with brine, dried, filtered and concentrated. Ethyl-2-{[(4-fluorobenzyl)methylamino]-methyl}-1-cyclopropane carboxylate is obtained.

6.1 ml of diisobutylaluminum hydride in cyclohexane are added drop-wise to a solution of 540 mg of ethyl 2-{[(4-fluorobenzyl)methylamino]methyl}-1-cyclopropane carboxylate in 10 ml of CH₂Cl₂ at −78°. The solution obtained is stirred for 2 hours prior to aq. workup with Rochelle's salt. The reaction mixture obtained is extracted with EtOAc and the combined organic layers obtained are dried, filtered and concentrated.

2-{[(4-fluorobenzyl)methylamino]methyl}-1-cyclopropyl methyl alcohol is obtained.

49 μl of methanesulfonyl chloride are added to a solution of 100 mg of 2-{[(4-fluorobenzyl)methylamino]methyl}-1-cyclopropyl methyl alcohol and 0.12 ml of Et₃N in 4 ml of CH₂Cl₂ at 0°. After 30 minutes the reaction mixture obtained is concentrated. The residue obtained is dissolved in 4 ml of DMF, 585 mg of NaN₃ are added and the mixture is heated at 75° for 4 hours. The reaction mixture obtained is allowed to cool, poured into 10 ml of H₂O and extracted 5 times with 15 ml of EtOAc. The combined organic layers obtained are dried, filtered and concentrated.

2-{[(4-fluorobenzyl)methylamino]methyl}-1-cyclopropyl methylazide is obtained.

79 mg of triphenylphosphine are added to a solution of 75 mg of 2-{[(4-fluorobenzyl)-methylamino]methyl}-1-cyclopropyl methyl azide in THF and the solution obtained is heated at reflux for 2 hours. 1 drop of H₂O is added and the mixture obtained is heated at reflux for a further 2 hours. The reaction mixture obtained is cooled and acidified by addition of 1N HCl. The mixture is concentrated and the residue obtained is triturated with diethyl ether. 2-{[(4-fluorobenzyl)methylamino]methyl}-1-cyclopropyl methylamine is obtained.

Et₃N (2.5 equiv) is added to 2-{[(4-fluorobenzyl)methylamino]methyl}-1-cyclopropyl methylamine (1.0 equiv) and phenyl 3-ethyl-5-(1-methyl-1H-tetrazol-5-yl)phenyl-carbamate (1.1 equiv) in ACN and the reaction mixture obtained is stirred at ambient temperature for 18 hours and concentrated.

1-(2-{[(4-fluorobenzyl)methylamino]methyl}-cyclopropylmethyl)-3-[3-(1-methyl-1H-tetrazol-5-yl)phenyl]-urea is obtained.

MS ESI (M+H)⁺=424

### Example 16

### 1-{(E)-4-[(4-Fluorobenzyl)-methylamino]but-2-enyl}-3-[3-(1-methyl-1H-tetrazol-5-yl)phenyl)-urea

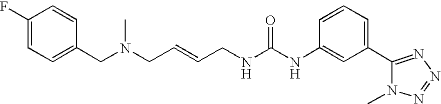

A mixture of 56 mg of (E)-N-(4-fluorobenzyl)-N-methyl-but-2-ene-1,4-diamine and 70 mg of phenyl 3-(1-methyl-1H-tetrazol-5-yl)-phenylcarbamate in 2 ml of anhydrous DMSO is stirred for 24 hours at ambient temperature. The mixture obtained is partitioned between saturated aq. sodium hydrogen carbonate solution and EtOAc, the aq. layer obtained is extracted 3 times with EtOAc. The combined organic layers obtained are dried, filtered and concentrated.

1-{(E)-4-[(4-Fluorobenzyl)methylamino]-but-2-enyl}-3-[3-(1-methyl-1H-tetrazol-5-yl)phenyl)-urea is obtained.

MS ESI (M+H)⁺=410

The following compound is prepared analogously as described in example 16:

### Example 17

### 1-[3,5-Bis-(1-methyl-1H-tetrazol-5-yl)-phenyl]-3-{(E)-4-[(3,4-dimethyl benzyl)-methylamino]-but-2-enyl}-urea

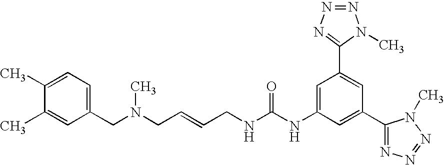

MS ESI (M+H)⁺=502.22

### Example 18

### (±)-1-{4-[(4-Fluorobenzyl)-methylamino]-3-hydroxybutyl}-3-[3-(1-methyl-1H-tetrazol-5-yl)phenyl]-urea

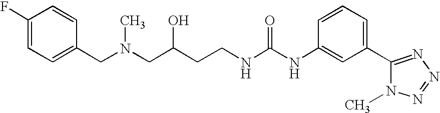

164 μl of Et₃N are added to a mixture of 108 mg of (±)-4-amino-1-[(4-fluorobenzyl)methylamino]-butan-2-ol bis-trifluoroacetate and 70 mg of phenyl-3-(1-methyl-1H-tetrazol-5-yl)-phenylcarbamate in 2 ml of anhydrous DMSO at ambient temperature. After stirring for 24 hours, the mixture obtained is partitioned between saturated aq. sodium hydrogen carbonate solution and EtOAc, and the aq. layer obtained is extracted 3 times with EtOAc. The combined organic layers obtained are dried, filtered and concentrated.

(±)-1-{4-[(4-Fluorobenzyl)methylamino]-3-hydroxy-butyl}-3-[3-(1-methyl-1H-tetrazol-5-yl)phenyl]-urea is obtained.

MS ESI (M+H)⁺=428

### Example 19

### 1-{4-[(4-Fluorobenzyl)-methylamino]butyl}-3-[3-((1-methyl-1H-tetrazol-5-yl)-5-thiophen-2-yl)-phenyl]-urea

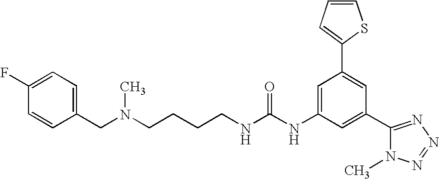

0.63 ml of tri-n-butyl(2-thienyl)tin and 130 mg of tetrakis(triphenylphosphine)palladium(0) are added to 320 mg of 3-bromo-5-nitrophenyl)-1-methyl-1H-tetrazole in 5 ml toluene and heated under reflux for 3 hours. The reaction mixture obtained is concentrated under reduced pressure. 1-methyl-5-(3-nitro-5-thiophen-2-yl)-1H-tetrazole is obtained.

A catalytic amount of palladium on carbon (10%) is added to 280 mg 1-methyl-5-(3-nitro-5-thiophen-2-yl)-1H-tetrazole in 3 ml MeOH and stirred under a hydrogen atmosphere for 30 minutes. The reaction mixture obtained is filtered under nitrogen through fiberglass filter paper and the filtrate obtained is concentrated. 3-(1-methyl-1H-tetrazol-5-yl)-5-thiophen-2-yl-phenylaniline is obtained.

0.13 ml of phenyl chloroformate are added drop-wise to 257 mg of 3-(1-methyl-1H-tetrazol-5-yl)-5-thiophen-2-yl-phenylaniline and 0.12 ml of 2,6-lutidine in 4 ml of THF and the reaction mixture obtained is stirred for 1 hour at ambient temperature. The reaction mixture obtained is diluted with EtOAc and 0.1M HCl is added. The organic layer obtained is isolated and washed with 0.1M HCl (2×), brine, dried (MgSO₄), filtered and concentrated under reduced pressure

Phenyl 3-(1-methyl-1H-tetrazol-5-yl)-5-(thiophen-2-yl)-phenylcarbamate is obtained.

0.03 ml of Et₃N are added to 27 mg of N-1-(4-fluorobenzyl)-N-1-methylbutane-1,4-diamine. 2HCl and 36 mg of phenyl 3-(1-methyl-1H-tetrazol-5-yl)-5-(thiophen-2-yl)-phenylcarbamate in 1 ml of ACN. The solution obtained is stirred at ambient temperature for 18 hours and concentrated.

1-{4-[(4-Fluorobenzyl)methylamino]butyl}-3-[3-((1-methyl-1H-tetrazol-5-yl)-5-thiophen-2-yl)phenyl]urea is obtained

MS ESI (M+H)⁺=494.

### Example 20

### 1-{4-[(4-Fluorobenzyl)-methylamino]-butyl}-3-[5-(1-methyl-1H-tetrazol-5-yl)-biphenyl-3-yl]-urea

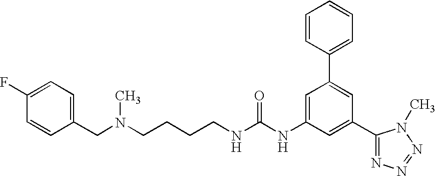

142 mg of phenyl boronic acid, 369 mg of potassium carbonate, 341 mg of tetrabutylammonium acetate and 0.5 mg of palladium (II) acetate are added to 274 mg of 3-bromo-5-nitrophenyl-1-methyl-1H-tetrazole in 1 ml of H₂O and heated at 70° C. for 1 hour. The reaction mixture obtained is diluted with H₂O and EtOAc and the layers obtained are separated. The aq. phase obtained is extracted twice with EtOAc and the combined organic extracts are washed with brine, dried over MgSO₄ and concentrated. 1-Methyl-5-(5-nitro-biphenyl-3-yl)-1H-tetrazole is obtained.

A catalytic amount of palladium on carbon (10%) is added to 264 mg of 1-methyl-5-(5-nitro-biphenyl-3-yl)-1H-tetrazole in 5 ml MeOH and 10 ml EtOAc and stirred under a hydrogen atmosphere for 3 hours. The reaction mixture obtained is through celite and the filtrate is concentrated. 5-(1-Methyl-1H-tetrazol-5-yl)-biphenyl-3-ylamine is obtained.

0.11 ml of phenyl chloroformate is added drop-wise to 216 mg of 5-(1-methyl-1H-tetrazol-5-yl)-biphenyl-3-ylamine and 145 mg of sodium hydrogen carbonate in 5 ml of THF at 0° C. and the reaction mixture obtained is stirred for 2 hours at ambient temperature. The reaction mixture is diluted with EtOAc and saturated aq. sodium hydrogen carbonate solution is added. The aq. phase obtained is extracted twice with EtOAc and the combined organic extracts are washed with brine, dried and concentrated.

[5-(1-Methyl-1H-tetrazol-5-yl)-biphenyl-3-yl]-carbamic acid phenyl ester is obtained.

0.09 ml of Et₃N are added to 61 mg of N-1-(4-fluorobenzyl)-N-1-methylbutane-1,4-diamine. 2HCl and 80 mg of [5-(1-methyl-1H-tetrazol-5-yl)-biphenyl-3-yl]-carbamic acid phenyl ester in 1 ml of DMSO. The solution obtained is heated in a microwave at 80° C. for 5 minutes. The reaction mixture obtained is diluted with H₂O and EtOAc and the layers obtained are separated. The aq. phase is extracted twice with EtOAc and the combined organic extracts obtained are washed with water then brine, dried and concentrated.

1-{4-[(4-Fluoro-benzyl)-methyl-amino]-butyl}-3-[5-(1-methyl-1H-tetrazol-5-yl)-biphenyl-3-yl]-urea is obtained.

MS APCI (M−H)⁺=486.41.

The following compounds are prepared analogously as described in Example 20:

### Example 21

### 1-[3-(1-Benzyl-1H-pyrazol-4-yl)-5-(1-methyl-1H-tetrazol-5-yl)-phenyl]-3-{4-[(4-fluorobenzyl)-methylamino]-butyl}-urea

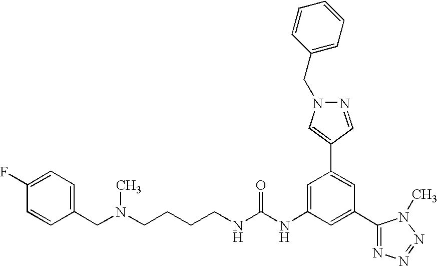

MS APCI (M+H)⁺=568.23.

### Example 22

### 1-{4-[(4-Fluoro-benzyl)-methylamino]-butyl}-3-[3-furan-2-yl-5-(1-methyl-1H-tetrazol-5-yl)-phenyl]-urea

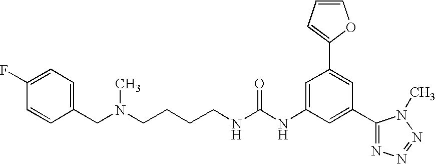

MS APCI (M+H)⁺=478.22.

### Example 23

### 1-[3-(2,3-Dihydro-1H-indol-5-yl)-5-(1-methyl-1H-tetrazol-5-yl)-phenyl]-3-{4-[(4-fluorobenzyl)-methylamino]-butyl}-urea

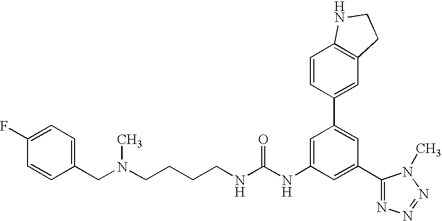

MS APCI (M+H)⁺=529.20.

### Example 24

### N-[3′-(3-{4-[(3,4-Dimethylbenzyl)-methylamino]-butyl}-ureido)-5′-(1-methyl-1H-tetrazol-5-yl)-biphenyl-3-yl]-]-acetamide trifluoroacetate

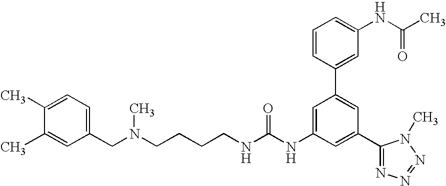

MS APCI (M+H)⁺=555.

### Example 25

### N-[3′-(3-{4-[(4-Fluorobenzyl)-methylamino]-butyl}-ureido)-5′-(1-methyl-1H-tetrazol-5-yl)-biphenyl-3-yl]-]-acetamide hydrochloride

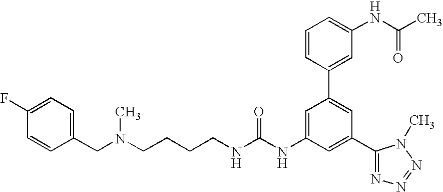

MS APCI (M+H)⁺=545.

### Example 26

### 1-{4-[(4-Fluorobenzyl)-methylamino]-butyl}-3-[3-(1-methyl-1H-tetrazol-5-yl)-5-pyridin-4-yl-phenyl]-urea

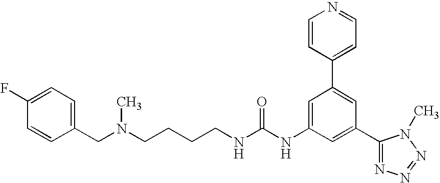

MS APCI (M+H)⁺=489.

### Example 27

### 1-{4-[(4-Fluorobenzyl)-methylamino]-butyl}-3-[3-(1-methyl-1H-tetrazol-5-yl)-5-pyridin-3-yl-phenyl]-urea

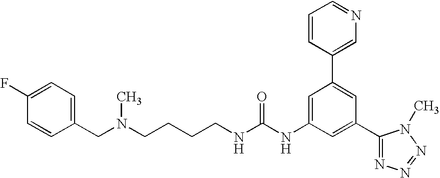

MS APCI (M+H)⁺=489.

### Example 28

### 3-(3-{4-[(3,4-Dimethylbenzyl)-methylamino]-butyl}-ureido)-N-methyl-5-(1-methyl-1H-tetrazol-5-yl)-benzamide

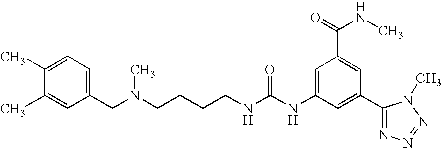

64.5 ml of a 2M solution of oxalyl chloride in DCM are added to a suspension of 15 g of 5-nitro isophthalic acid monomethyl ester in 200 ml of DCM and a few drops of DMF. After 2 hours stirring at ambient temperature the solvent is removed and the residue obtained is co-concentrated twice with toluene. The residue obtained is dissolved in 150 ml of THF and added dropwise to 100 ml of an ice-cooled 2M solution of methylamine in THF. The mixture obtained is stirred at ambient temperature overnight. The solid obtained is filtered and washed with THF, the filtrate obtained is concentrated and the solid obtained is washed with ether and dried.

N-Methyl-5-nitro-isophthalamic acid methyl ester is obtained.

12.2 ml of triflic anhydride are added dropwise to an ice-cooled mixture of 4.61 g NaN₃ and 15.3 g N-methyl-5-nitro-isophthalamic acid methyl ester in 300 ml ACN under an inert atmosphere. The mixture obtained is stirred at ambient temperature for 90 minutes and partitioned between saturated aq. sodium carbonate and EtOAc. The aq. layer obtained is extracted three times with EtOAc. The combined organic layers obtained are dried and concentrated.

3-(1-Methyl-1H-tetrazol-5-yl)-5-nitro-benzoic acid methyl ester is obtained.

1.16 g of LiOH are added to a solution of 11.7 g of 3-(1-methyl-1H-tetrazol-5-yl)-5-nitro-benzoic acid methyl ester in 100 ml. After stirring for 10 minutes at ambient temperature TLC indicates complete conversion. The reaction mixture obtained is partitioned between EtOAc and 0.1 M hydrochloric acid, the organic layer obtained is dried and concentrated.

3-(1-Methyl-1H-tetrazol-5-yl)-5-nitro-benzoic acid is obtained.

91.5 ml methyl amine (2M in THF) are added to a mixture of 9.13 g of 3-(1-methyl-1H-tetrazol-5-yl)-5-nitro-benzoic acid, 5.86 g N-hydroxybenzotriazole and 14.0 g 1-(3-dimethylaminopropyl)-3-ethylcarbodiimide hydrochloride in 100 ml of DMF. The mixture obtained is stirred for 20 hours at ambient temperature and partitioned between EtOAc and H₂O. The aq. layer obtained is extracted twice with EtOAc, the combined organic extracts obtained are washed with saturated aq. sodium hydrogen carbonate solution and brine, dried and concentrated.

N-methyl-3-(1-methyl-1H-tetrazol-5-yl)-5-nitro-benzamide is obtained.

A catalytic amount of palladium on carbon (10%) is added to 8.78 g of N-methyl-3-(1-methyl-1H-tetrazol-5-yl)-5-nitro-benzamide in 800 ml MeOH and stirred under a hydrogen atmosphere for 3 hours. TLC indicates complete consumption of the starting material and the suspension obtained is diluted with 1000 ml ACN. The catalyst is removed by filtration, the filter cake obtained is washed with ACN/EtOH 1:1 and the filtrate obtained is concentrated.

3-Amino-N-methyl-3-(1-methyl-1H-tetrazol-5-yl)-benzamide is obtained.

3.46 mL of phenylchloroformate are added dropwise to an ice-cooled suspension of 6.36 g of 3-amino-N-methyl-3-(1-methyl-1H-tetrazol-5-yl)-benzamide and 4.6 g of sodium carbonate in 300 ml of THF. After 30 minutes the ice bath is removed and the mixture obtained is stirred for additional 3 hours at ambient temperature. The mixture obtained is diluted with 500 ml of ACN and filtered. The filter cake obtained is washed with THF/ACN 1:1 and the filtrate obtained is concentrated.

[3-Methylcarbamoyl-5-(1-methyl-1H-tetrazol-5-yl)-phenyl]-carbamic acid phenyl ester is obtained.

A mixture of 3.0 g of [3-methylcarbamoyl-5-(1-methyl-1H-tetrazol-5-yl)-phenyl]-carbamic acid phenyl ester, 2.5 g of N-1-(3,4-dimethylbenzyl)-N-1-methylbutane-1,4-diamine dihydrochloride and 3.68 mL of triethylamine in 25 ml DMSO is heated to 100° C. for 10 minutes under an inert atmosphere. The reaction mixture obtained is cooled to ambient temperature and partitioned between EtOAc and H₂O. The aq. layer obtained is washed twice with EtOAc, the combined organic layers obtained are dried and concentrated. 3-(3-{4-[(3,4-Dimethyl-benzyl)-methyl-amino]-butyl}-ureido)-N-methyl-5-(1-methyl-1H-tetrazol-5-yl)-benzamide is obtained.

MS ESI (M+H)⁺=479.

The following compound is prepared analogously as described in Example 28:

### Example 29

### 3-(3-{4-[(4-Fluorobenzyl)-methylamino]-butyl}-ureido)-N-methyl-5-(1-methyl-1H-tetrazol-5-yl)-benzamide

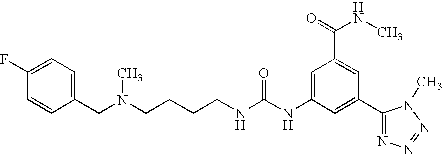

MS ESI (M+H)⁺=494.

### Example 30

### 1-{4-[(3,4-Dimethylbenzyl)-methylamino]-butyl}-3-[3-tetrazol-5-yl-methyl)phenyl]-urea trifluoroacetate

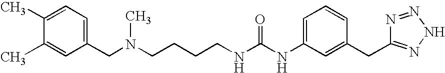

A mixture of 2.0 g of 3-(nitrophenyl)-acetonitrile, 882 mg of NaN₃, 2.78 g of ZnBr₂ and 30 ml of H₂O is heated in a sealed tube to 170° C. for 17 hours with efficient stirring. The cooled mixture obtained is acidified with 3N hydrochloric acid and extracted with EtOAc. The combined organic extracts obtained are concentrated and the residue is treated with 0.25 N NaOH under efficient stirring. The mixture obtained is filtered and the filter cake obtained is washed with 1N NaOH. The filtrate obtained is acidified by addition of 3N HCl, the solid obtained is filtered, washed with 3N HCl and dried. 5-(3-Nitro-benzyl)-tetrazole is obtained.

A mixture of 800 mg of 5-(3-nitro-benzyl)-tetrazole and a catalytic amount 10% palladium on charcoal in 50 ml of EtOH is stirred under an atmosphere of hydrogen at ambient temperature for 90 minutes. The reaction mixture obtained is filtered through a plug of celite, the filter cake obtained is washed with EtOH and the filtrate obtained concentrated.

3-(Tetrazol-5-ylmethyl)-aniline is obtained.

470 μL of phenyl chloroformate are added dropwise to an ice-cooled mixture of 640 mg of 3-(tetrazol-5-ylmethyl)-aniline and 620 mg of sodium carbonate. After stirring for 2 hours at 0° C., the mixture obtained is partitioned between saturated aq. sodium hydrogen carbonate solution and

EtOAc and the aq. layer obtained is extracted twice with EtOAc. The combined organic extracts obtained are washed with 10% citric acid, brine, dried and concentrated.

[3-(2H-Tetrazol-5-ylmethyl)-phenyl]-carbamic acid phenyl ester is obtained.

A mixture of 73 mg of [3-(2H-tetrazol-5-ylmethyl)-phenyl]-carbamic acid phenyl ester, 70 mg of N-1-(3,4-dimethylbenzyl)-N-1-methylbutane-1,4-diamine dihydrochloride and 63 μl of triethylamine in 1 ml of DMSO is heated to 100° C. for 10 minutes under an inert atmosphere. The reaction mixture obtained is cooled to ambient temperature and partitioned between EtOAc and H₂O. The aq. layer obtained is washed twice with EtOAc, the combined organic layers obtained are dried and concentrated.

MS ESI (M−H)⁺=534.

### Example 31

### N-[4-bromo-3-(3-{4-[(4-fluorobenzyl)methylamino]butyl}ureido)phenyl]acetamide

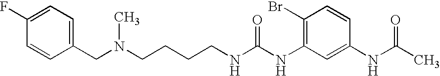

A mixture of 1.0 g of 4-bromo-3-nitroaniline in 20 ml of acetic anhydride is stirred at ambient temperature for 5 minutes. The reaction mixture obtained is concentrated.

N-(4-bromo-3-nitrophenyl)acetamide is obtained.

1.10 g of N-(4-bromo-3-nitrophenyl)acetamide are dissolved in 2 ml of MeOH. A catalytic amount of 10% palladium on activated charcoal is added and the mixture obtained is hydrogenated at ambient temperature and atmospheric pressure for 24 hours, filtered through celite and the filtrate obtained is concentrated. N-(3-amino-4-bromophenyl)acetamide is obtained.

63 mg of N-(3-amino-4-bromophenyl)acetamide are dissolved in DCM, 65 mg of pyridine and 65 mg phenyl chloroformate are added. After stirring at ambient temperature for 30 minutes, the mixture obtained is quenched with 1N HCl. After exhaustive extraction with DCM, the combined organic layers obtained are concentrated.

(5-acetylamino-2-bromophenyl)carbamic acid phenyl ester is obtained.

24 mg of triethylamine are added to a solution of 31 mg of N-(4-fluorobenzyl)-N-methylbutane-1,4-diamine dihydrochloride and 40 mg of (5-acetylamino-2-bromophenyl)carbamic acid phenyl ester in DCM The mixture obtained is stirred at ambient temperature for 24 hours. Saturated aq. sodium hydrogen carbonate solution is added and exhaustive extraction with DCM is carried out. The combined organic extracts obtained are concentrated.

N-[4-bromo-3-(3-{4-[(4-fluorobenzyl)methylamino]butyl}ureido)phenyl]acetamide is obtained.

MS ESI (M+H)⁺=467.10

The following compound is prepared analogously as described in Example 31:

### Example 32

### N-[4-Chloro-3-(3-{4-[(4-fluorobenzyl)-methylamino]-butyl}-ureido)-phenyl]-acetamide

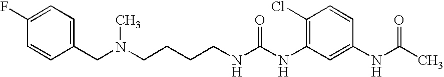

MS ESI (M+H)⁺=421.18

### Example 33

### 5-(3-{4-[(4-Fluorobenzyl)-methylamino]-butyl}-ureido)-N,N,N′,N′-tetramethyl-isophthalamide

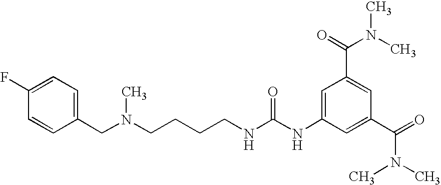

To a solution of 54 mg of N-(4-fluorobenzyl)-N-methylbutane-1,4-diamine dihydrochloride and 70 mg of (3,5-bis-dimethylcarbamoylphenyl)carbamic acid phenyl ester in 2 ml DCM are added 79 μl of triethylamine. The mixture obtained is stirred at ambient temperature for 24 hours. After adding saturated aq. sodium hydrogen carbonate solution and exhaustive extraction with DCM, the combined organic extracts obtained are concentrated. 5-(3-{4-[(4-fluorobenzyl)methylamino]butyl}ureido)-N,N,N′,N′-tetramethylisophthalamide is obtained.

MS ESI (M+H)⁺=472.26

The following compound is prepared analogously as described in Example 33:

### Example 34

### 5-(3-{4-[(3,4-Dimethylbenzyl)-methylamino]-butyl}-ureido)-N,N,N′,N′-tetramethyl-isophthalamide

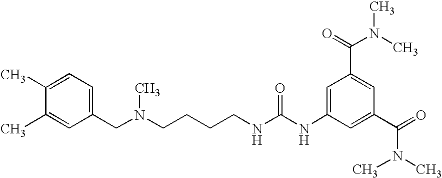

MS ESI (M+H)⁺=482.28

### Example 35

### 1-{4-[N-(4-fluoro-3-methyl-benzyl)-N-methylamino]butyl}-3-[3-(1-methyl-1H-tetrazol-5-yl)phenyl]urea

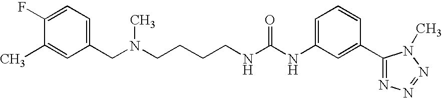

A mixture of 10 ml of N-benzylmethylamine, 27.3 g of N-(4-bromobutyl)phthalimide and 126 mg of potassium iodide in 150 ml MeOH is stirred at 80° C. for 4 hours. After cooling, the mixture obtained is partitioned between saturated aq. sodium hydrogen carbonate solution and EtOAc. After exhaustive extraction with EtOAc, the combined organic extracts obtained are washed with brine, dried and concentrated.

2-[4-(benzylmethylamino) butyl]isoindole-1,3-dione is obtained.

15 g of 2-[4-(benzylmethylamino) butyl]isoindole-1,3-dione in 200 ml MeOH are treated with 4.67 g of hydrazine hydrate at ambient temperature. After stirring for 3 hours, another equivalent of hydrazine hydrate is added and stirring continues for 3 hours. After evaporation of the solvent, the residue obtained is taken up in EtOAc and filtered. The filtrate obtained is concentrated and treated with boiling MeOH/2N NaOH (1:1) for 2 hours. Upon cooling, the mixture obtained is partitioned between brine and Et₂₀. After exhaustive extraction with Et₂₀, the combined organic extracts obtained are dried and concentrated. N-benzyl-N-methylbutane-1,4-diamine is obtained.

5.87 g of N-benzyl-N-methylbutane-1,4-diamine and 3.07 g of [3-(1-methyl-1H-tetrazol-5-yl)phenyl]carbamic acid phenyl ester in 50 ml of DMSO are stirred at ambient temperature for 6 hours. After partitioning with saturated aq. sodium hydrogen carbonate solution and EtOAc and exhaustive extraction with EtOAc, the combined organic extracts obtained are washed with brine, dried and concentrated.

1-[4-(benzylmethyl-amino)butyl]-3-[3-(1-methyl-1H-tetrazol-5-yl)phenyl]urea is obtained. 3.32 g of 1-[4-(benzylmethyl-amino)butyl]-3-[3-(1-methyl-1H-tetrazol-5-yl)phenyl]urea and a catalytic amount of 10% palladium on activated charcoal in 20 ml of 2N HCl in Et₂O and 5 ml of MeOH are hydrogenated at 70° C. at atmospheric pressure for 4 hours. After cooling and filtering through celite, the solution obtained is concentrated to give the crude hydrochloride salt, which is converted into the free base with a mixture of saturated aq. sodium hydrogen carbonate solution and ammonium hydroxide.

1-(4-methylamino-butyl)-3-[3-(1-methyl-1H-tetrazol-5-yl)-phenyl]urea is obtained.

55 mg of sodium triacetoxyborohydride are added to a stirred solution of 50 mg of 1-(4-methylamino-butyl)-3-[3-(1-methyl-1H-tetrazol-5-yl)-phenyl]-urea and 25 μl of 4-fluoro-3-methylbenzaldehyde in 1 ml of THF. After 4 hours of stirring at ambient temperature, the mixture obtained is partitioned between ammonium hydroxide and DCM. After exhaustive extraction with DCM, the combined organic extracts obtained are dried and concentrated. 1-{4-[N-(4-fluoro-3-methylbenzyl)-N-methylamino]butyl}-3-[3-(1-methyl-1H-tetrazol-5-yl)phenyl]urea is obtained.

MS ESI (M+H)⁺=426.28

The following compounds are prepared analogously as described in Example 35:

### Example 36

### 1-{4-[(3-Fluoro-4-methyl-benzyl)-methylamino]-butyl}-3-[3-(1-methyl-1H-tetrazol-5-yl)-phenyl]-urea

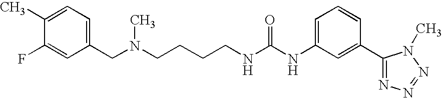

MS ESI (M+H)⁺=426.28

### Example 37

### 1-{4-[(3,4-Difluorobenzyl)-methylamino]-butyl}-3-[3-(1-methyl-1H-tetrazol-5-yl)phenyl]-urea

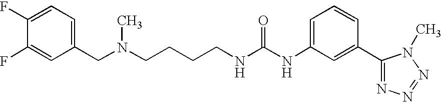

MS ESI (M+H)⁺=430.25

### Example 38

### 1-[3,5-Bis-(1-methyl-1H-tetrazol-5-yl)-phenyl]-3-{4-[(4-fluoro-3,5-dimethylbenzyl)-methylamino]-butyl}-urea

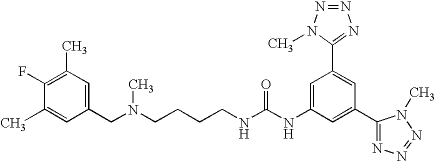

MS ESI (M+H)⁺=522.34

### Example 39

### 1-[3,5-Bis-(1-methyl-1H-tetrazol-5-yl)-phenyl]-3-{4-[(4-fluoro-3-methyl-benzyl)methylamino]-butyl}-urea

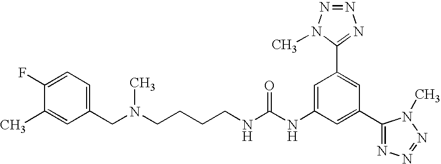

MS ESI (M+H)⁺=508.30

### Example 40

### 1-[3,5-Bis-(1-methyl-1H-tetrazol-5-yl)-phenyl]-3-{4-[(3-fluoro-4-methyl-benzyl)methylamino]-butyl}-urea

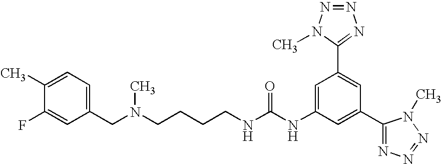

MS ESI (M+H)⁺=530.26

### Example 41

### 1-[3,5-Bis-(1-methyl-1H-tetrazol-5-yl)-phenyl]-3-[4-(indan-5-yl-methyl-methylamino)-butyl]-urea

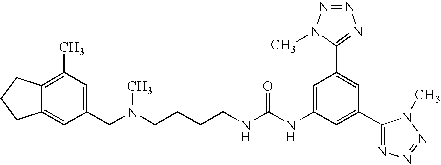

MS ESI (M+H)⁺=516.29

### Example 42

### 1-{4-[Cyclopropyl-(4-fluorobenzyl)-amino]-butyl}-3-[3-(1-methyl-1H-tetrazol-5-yl)-phenyl]-urea

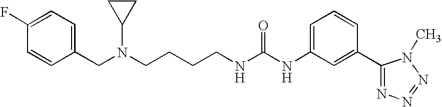

A mixture of 88 mg N-cyclopropyl-N-(4-fluorobenzyl)butane-1,4-diamine and 100 mg of [3-(1-methyl-1H-tetrazol-5-yl)-phenyl]carbamic acid phenyl ester in 2 ml of DMSO is stirred at ambient temperature for 24 hours. After quenching with saturated aq. sodium hydrogen carbonate solution and exhaustive extraction with EtOAc, the combined organic extracts obtained are dried and concentrated. 1-{4-[cyclopropyl-(4-fluorobenzyl)amino]butyl}-3-[3-(1-methyl-1H-tetrazol-5-yl)phenyl]urea is obtained.

MS ESI (M+H)⁺=438.25

The following compound is prepared analogously as described in Example 42:

### Example 43

### 1-{4-[Cyclopropyl-(3,4-dimethylbenzyl)-amino]-butyl}-3-[3-(1-methyl-1H-tetrazol-5-yl)-phenyl]-urea

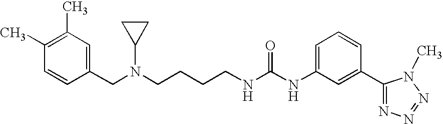

MS ESI (M+H)⁺=448.26

### Example 44

### 3-(3-{4-[(4-Fluorobenzyl)-methylamino]-butyl}-ureido)-N,N-dimethyl-5-(1-methyl-1H-tetrazol-5-yl)-benzamide

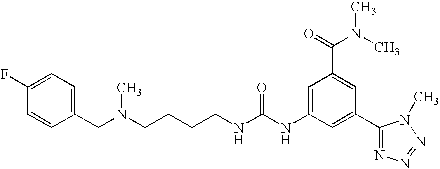

24.3 g of SnCl₂ dihydrate are added to a solution of 7.3 g of methyl 3-(1-methyl-1H-tetrazol-5-yl)-5-nitrobenzoate in 150 ml MeOH. After stirring under reflux for 2 hours, the mixture obtained is partitioned between saturated aq. sodium hydrogen carbonate solution and EtOAc. After exhaustive extraction with EtOAc, the combined organic extracts obtained are washed twice with brine, dried and concentrated under reduced pressure.

Methyl 3-amino-5-(1-methyl-1H-tetrazol-5-yl)-benzoate is obtained.

1.94 g of triethylamine and 5.44 g of diphenylphosphoryl azide are added to a solution of 3.89 g of 5-(tert-butoxycarbonyl-methyl-amino)pentanoic acid in 100 ml of toluene. After 2 hours of stirring at 110° C., the mixture obtained is cooled to 25° C. and 3.80 g of methyl 3-amino-5-(1-methyl-1H-tetrazol-5-yl)-benzoate are added. The mixture obtained is re-heated to 110° C. and stirred for 2 hours, cooled, and the mixture obtained is partitioned between saturated aq. sodium hydrogen carbonate solution and EtOAc. After exhaustive extraction with EtOAc, the combined organic extracts obtained are washed with saturated aq. sodium hydrogen carbonate solution, dried and concentrated.

Methyl 3-{3-[4-(tert-butoxycarbonyl-methylamino)butyl]ureido}-5-(1-methyl-1H-tetrazol-5-yl)benzoate is obtained.

2.8 g of methyl 3-{3-[4-(tert-butoxycarbonyl-methylamino)butyl]ureido}-5-(1-methyl-1H-tetrazol-5-yl)benzoate are treated with 1.27 g of LiOH hydrate in 300:50:50 ml THF/MeOH/H₂O for 2 hours at ambient temperature. The mixture obtained is acidified with 1N HCl. After exhaustive extraction with EtOAc, the combined organic extracts obtained are washed with H₂O, dried and concentrated. 3-{3-[4-(tert-Butoxycarbonyl-methylamino)butyl]ureido}-5-(1-methyl-1H-tetrazol-5-yl)-benzoic acid is obtained.

200 mg of 3-{3-[4-(tert-Butoxycarbonyl-methylamino)butyl]ureido}-5-(1-methyl-1H-tetrazol-5-yl)-benzoic acid are dissolved in 2 ml of DMF and treated with 60 mg of 1-hydroxybenzotriazole, 174 mg of 1-(3-dimethylaminopropyl)-3-ethylcarbodiimide hydrochloride, 147 μl of N-methylmorpholine and 270 μl of a 2M solution of dimethylamine in THF. After stirring at ambient temperature for 24 hours, the mixture obtained is partitioned between saturated aq. sodium hydrogen carbonate solution and DCM. After exhaustive extraction with DCM, the combined organic extracts obtained are concentrated. The residue obtained is treated with a 4M solution of HCl in dioxane at ambient temperature for 3 hours and concentrated.

N,N-dimethyl-3-[3-(4-methylamino-butyl)-ureido]-5-(1-methyl-1H-tetrazol-5-yl)benzamide hydrochloride is obtained.

41 ul of Triethylamine and 98 mg of sodium triacetoxyborohydride are added to a stirred solution of 60 mg of N,N-dimethyl-3-[3-(4-methylamino-butyl)-ureido]-5-(1-methyl-1H-tetrazol-5-yl)benzamide hydrochloride and 28 mg of 4-fluorobenzaldehyde in 4 ml of DCM. After stirring at ambient temperature for 24 hours, the mixture obtained is partitioned between saturated aq. sodium hydrogen carbonate solution and DCM. After exhaustive extraction with DCM, the combined organic extracts obtained are concentrated. 3-(3-{4-[(4-Fluorobenzyl)methylamino]butyl}ureido)-N,N-dimethyl-5-(1-methyl-1H-tetrazol-5-yl)benzamide is obtained.

MS ESI (M+H)⁺=483.17

The following compounds are prepared analogously as described in Example 44:

### Example 45

### N-Ethyl-3-(3-{4-[(4-fluorobenzyl)-methylamino]-butyl}-ureido)-5-(1-methyl-1H-tetrazol-5-yl)-benzamide

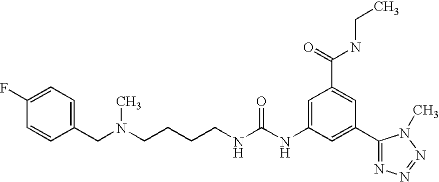

MS ESI (M+H)⁺=483.23

### Example 46

### 3-(3-{4-[(4-Fluorobenzyl)-methylamino]-butyl}-ureido)-N-isopropyl-5-(1-methyl-1H-tetrazol-5-yl)-benzamide

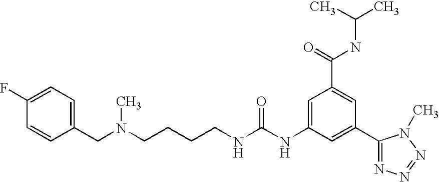

MS ESI (M+H)⁺=497.23

### Example 47

### 1-{4-[(4-Fluorobenzyl)-methylamino]-butyl}-3-[3-(1-methyl-1H-tetrazol-5-yl)-5-(piperidine-1-carbonyl)-phenyl]-urea

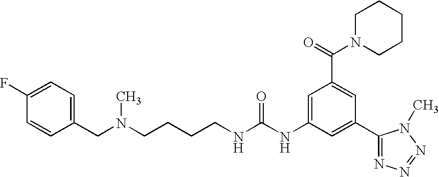

MS ESI (M+H)⁺=523.23

### Example 48

### 1-{4-[(4-Fluorobenzyl)-methylamino]-butyl}-3-[3-(1-methyl-1H-tetrazol-5-yl)-5-(morpholine-4-carbonyl)-phenyl]-urea

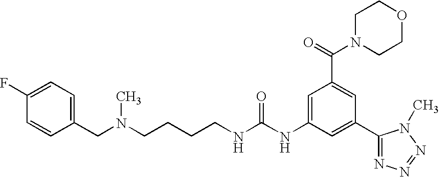

MS ESI (M+H)⁺=547.12

### Example 49

### 1-{4-[(4-Fluorobenzyl)-methylamino]-butyl}-3-[3-(4-methyl-piperazine-1-carbonyl)-5-(1-methyl-1H-tetrazol-5-yl)-phenyl]-urea

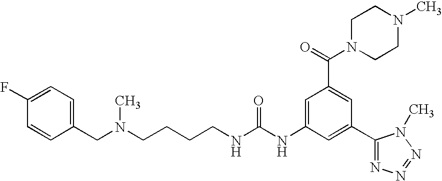

MS ESI (M+H)⁺=538.24

### Example 50

### 1-{4-[(4-Fluorobenzyl)-methylamino]-butyl}-3-[3-(1-methyl-1H-tetrazol-5-yl)-5-(pyrrolidine-1-carbonyl)-phenyl]-urea

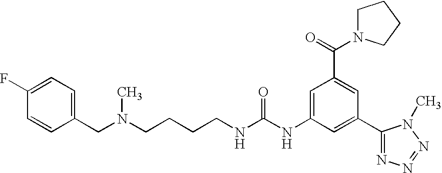

MS ESI (M+H)⁺=509.18

### Example 51

### N-Cyclopropylmethyl-3-(3-{4-[(4-fluorobenzyl)-methylamino]-butyl}-ureido)-5-(1-methyl-1H-tetrazol-5-yl)-benzamide

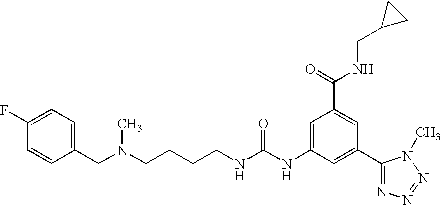

MS ESI (M+H)⁺=509.23

### Example 52

### 3-(3-{4-[(4-Fluorobenzyl)-methylamino]-butyl}-ureido)-5-(1-methyl-1H-tetrazol-5-yl)-N-phenyl-benzamide

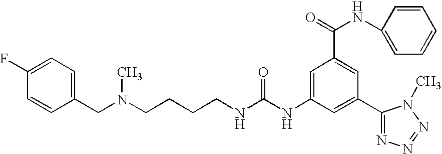

MS ESI (M+H)⁺=531.22

### Example 53

### 3-(3-{4-[(4-Fluorobenzyl)-methylamino]-butyl}-ureido)-5-(1-methyl-1H-tetrazol-5-yl)-N-pyridin-2-ylmethyl-benzamide

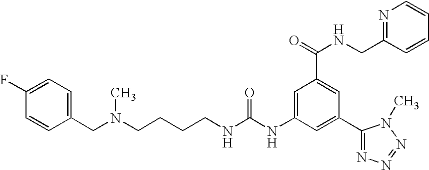

MS ESI (M+H)⁺=546.13

### Example 54

### N-Benzyl-3-(3-{4-[(4-fluorobenzyl)-methylamino]-butyl}-ureido)-5-(1-methyl-1H-tetrazol-5-yl)-benzamide

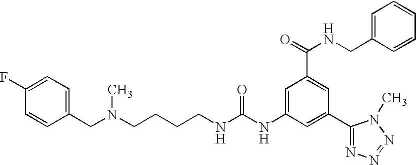

MS ESI (M+H)⁺=545.21

### Example 55

### N-(2-Dimethylaminoethyl)-3-(3-{4-[(4-fluorobenzyl)-methylamino]-butyl}-ureido)-5-(1-methyl-1H-tetrazol-5-yl)-benzamide

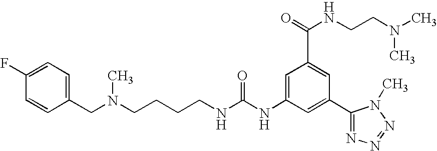

MS ESI (M+H)⁺=526.29

### Example 56

### 3-(3-{4-[(3,4-Dimethylbenzyl)-methylamino]-butyl}-ureido)-N,N-dimethyl-5-(1-methyl-1H-tetrazol-5-yl)-benzamide

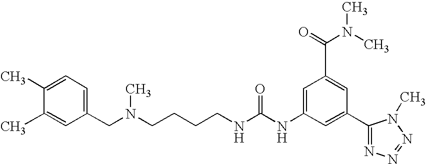

MS ESI (M+H)⁺=493.26

### Example 57

### N-Ethyl-3-(3-{4-[(3,4-Dimethylbenzyl)-methylamino]-butyl}-ureido)-5-(1-methyl-1H-tetrazol-5-yl)-benzamide

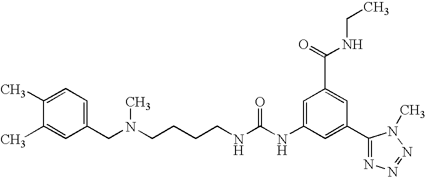

MS ESI (M+H)⁺=493.27

### Example 58

### 3-(3-{4-[(3,4-Dimethylbenzyl)-methylamino]-butyl}-ureido)-N-isopropyl-5-(1-methyl-1H-tetrazol-5-yl)-benzamide

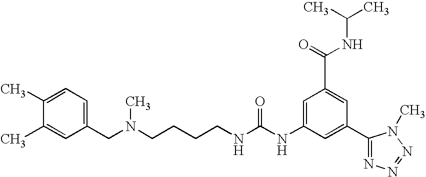

MS ESI (M+H)⁺=507.29

### Example 59

### 1-{4-[(3,4-Dimethylbenzyl)-methylamino]-butyl}-3-[3-(1-methyl-1H-tetrazol-5-yl)-5-(piperidine-1-carbonyl)-phenyl]-urea

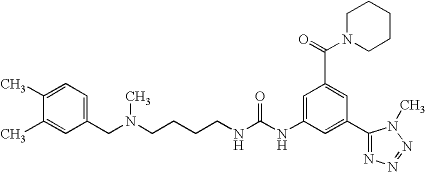

MS ESI (M+H)⁺=533.27

### Example 60

### 1-{4-[(3,4-Dimethylbenzyl)-methylamino]-butyl}-3-[3-(1-methyl-1H-tetrazol-5-yl)-5-(morpholine-4-carbonyl)-phenyl]-urea

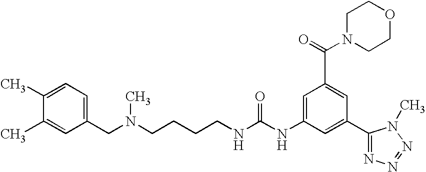

MS ESI (M+H)⁺=535.26

### Example 61

### 1-{4-[(3,4-Dimethylbenzyl)-methylamino]-butyl}-3-[3-(4-methyl-piperazine-1-carbonyl)-5-(1-methyl-1H-tetrazol-5-yl)-phenyl]-urea

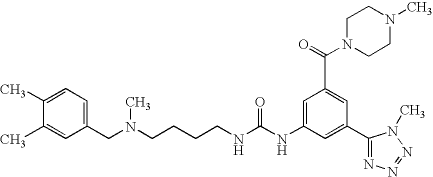

MS ESI (M+H)⁺=548.30

### Example 62

### 1-{4-[(3,4-Dimethylbenzyl)-methylamino]-butyl}-3-[3-(1-methyl-1H-tetrazol-5-yl)-5-(pyrrolidine-1-carbonyl)-phenyl]-urea

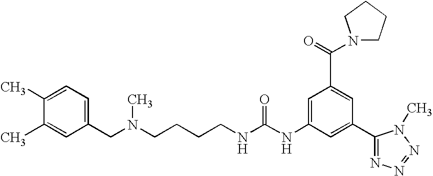

MS ESI (M+H)⁺=541.24

### Example 63

### N-Cyclopropylmethyl-3-(3-{4-[(3,4-dimethylbenzyl)-methylamino]-butyl}-ureido)-5-(1-methyl-1H-tetrazol-5-yl)-benzamide

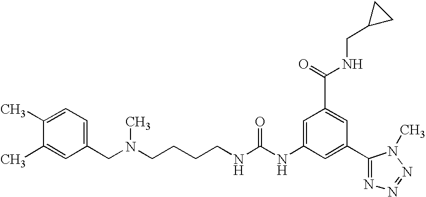

MS ESI (M+H)⁺=519.26

### Example 64

### 3-(3-{4-[(3,4-Dimethylbenzyl)-methylamino]-butyl}-ureido)-5-(1-methyl-1H-tetrazol-5-yl)-N-phenyl-benzamide

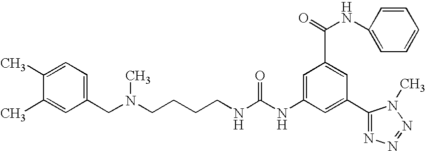

MS ESI (M+H)⁺=541.24

### Example 65

### N-Benzyl-3-(3-{4-[(3,4-dimethylbenzyl)-methylamino]-butyl}-ureido)-5-(1-methyl-1H-tetrazol-5-yl)-benzamide

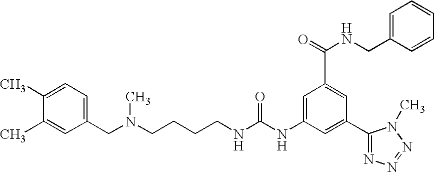

MS ESI (M+H)⁺=555.28

### Example 66

### 3-(3-{4-[(3,4-Dimethylbenzyl)-methylamino]-butyl}-ureido)-5-(1-methyl-1H-tetrazol-5-yl)-N-pyridin-2-ylmethyl-benzamide

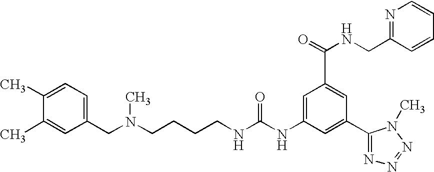

MS ESI (M+H)⁺=556.27

### Example 67

### N-(2-Dimethylaminoethyl)-3-(3-{4-[(3,4-dimethylbenzyl)-methylamino]-butyl}-ureido)-5-(1-methyl-1H-tetrazol-5-yl)-benzamide

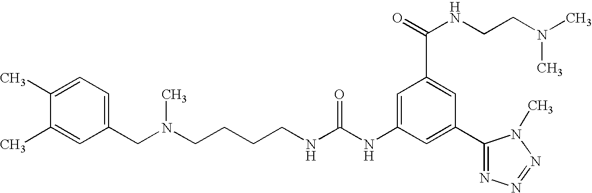

MS ESI (M+H)⁺=536.22

### Example 68

### 3-{3-[4-(Indan-5-yl-methyl-methylamino)-butyl]-ureido}-N,N-dimethyl-5-(1-methyl-1H-tetrazol-5-yl)-benzamide

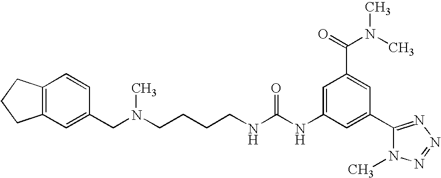

MS ESI (M+H)⁺=505.31

### Example 69

### 3-(3-{4-[(4-Fluoro-3-methoxy-benzyl)-methylamino]-butyl}-ureido)-N,N-dimethyl-5-(1-methyl-1H-tetrazol-5-yl)-benzamide

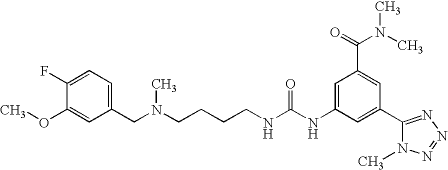

MS ESI (M+H)⁺=513.26

### Example 70

### 3-(3-{4-[(4-Fluoro-3-hydroxy-benzyl)-methylamino]-butyl}-ureido)-N,N-dimethyl-5-(1-methyl-1H-tetrazol-5-yl)-benzamide

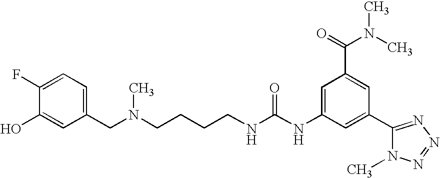

MS ESI (M+H)⁺=499.20

### Example 71

### 3-(3-{4-[(3-Cyclopropylmethoxy-4-fluorobenzyl)-methylamino]-butyl}-ureido)-N,N-dimethyl-5-(1-methyl-1H-tetrazol-5-yl)-benzamide

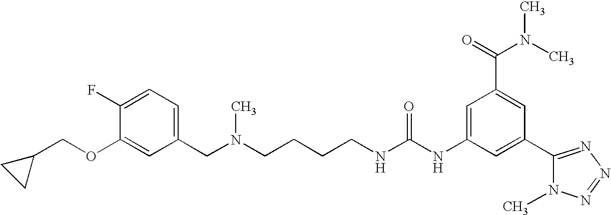

MS ESI (M+H)⁺=553.31

### Example 72

### t-Butyl-3-(3-{4-[(4-Fluorobenzyl)-methylamino]-butyl}-ureido)-5-(1-methyl-1H-tetrazol-5-yl)-benzoate

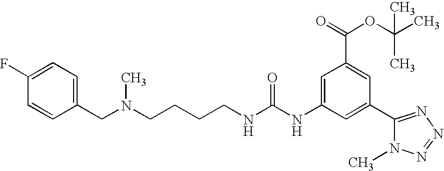

9.37 g of lithium hydroxide monohydrate are added to a solution of 14.7 g of methyl 3-(1-methyl-1H-tetrazol-5-yl)-5-nitrobenzoate in 180:60:60 ml THF/MeOH/H₂O. After stirring for 4 hours at ambient temperature, the mixture obtained is acidified with 1N HCl. After exhaustive extraction with EtOAc the combined organic extracts obtained are dried and concentrated.

3-(1-Methyl-1H-tetrazol-5-yl)-5-nitro-benzoic acid is obtained.

1.00 g of 3-(1-methyl-1H-tetrazol-5-yl)-5-nitro-benzoic acid, 312 mg of t-butanol, 795 mg of dicyclohexylcarbodiimide and 47 mg of DMAP in 20 mL of CH₂Cl₂ are stirred for 24 hours at ambient temperature. The mixture obtained is concentrated.

t-Butyl 3-(1-methyl-1H-tetrazol-5-yl)-5-nitro-benzoate is obtained.

Hydrogen gas is bubbled through a slurry of 880 mg of f-butyl 3-(1-methyl-1H-tetrazol-5-yl)-5-nitro-benzoate and 180 mg of palladium on activated charcoal in 40 ml of MeOH during 4 hours at ambient temperature. The mixture obtained is filtered through celite and concentrated.

t-Butyl 3-amino-5-(1-methyl-1H-tetrazol-5-yl)-benzoate is obtained.

To an ice-cold solution of 400 mg of t-butyl 3-amino-5-(1-methyl-1H-tetrazol-5-yl)-benzoate and 0.332 ml of pyridine in 20 ml of CH₂Cl₂, 0.262 ml of phenyl chloroformate are added. After stirring for 10 minutes at 5°, the mixture obtained is partitioned between saturated aq. sodium hydrogen carbonate solution and EtOAc. After exhaustive extraction with EtOAc the combined organic extracts obtained are dried and concentrated.

t-Butyl 3-(1-methyl-1H-tetrazol-5-yl)-5-[bis(phenoxycarbonyl)]-amino-benzoate is obtained.

A solution of 350 mg of N-1-(4-fluorobenzyl)-N-1-methylbutane-1,4-diamine. 2HCl, 694 mg of t-butyl 3-(1-methyl-1H-tetrazol-5-yl)-5-[bis(phenoxycarbonyl)]-amino-benzoate, and 0.508 ml of triethylamine in 5 ml of CH₂Cl₂ are stirred for 24 hours at ambient temperature. The mixture obtained is partitioned between saturated aq. sodium hydrogen carbonate solution and EtOAc. After exhaustive extraction with EtOAc the combined organic extracts obtained are dried and concentrated.

t-Butyl-3-(3-{4-[(4-Fluoro-benzyl)-methyl-amino]-butyl}-ureido)-5-(1-methyl-1H-tetrazol-5-yl)benzoate is obtained.

MS ESI (M+H)⁺=512.26

### Example 73

### 3-(3-{4-[(4-Fluorobenzyl)-methylamino]-butyl}-ureido)-5-(1-methyl-1H-tetrazol-5-yl)-benzoic acid hydrochloride

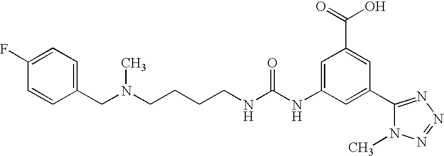

440 mg of t-butyl-3-(3-{4-[(4-Fluoro-benzyl)-methyl-amino]-butyl}-ureido)-5-(1-methyl-1H-tetrazol-5-yl)-benzoate is dissolved in 5 ml of dioxane and treated with 5 ml of 4N HCl in dioxane for 72 hours at ambient temperature. The mixture obtained is concentrated. 3-(3-{4-[(4-Fluoro-benzyl)-methylamino]-butyl}-ureido)-5-(1-methyl-1H-tetrazol-5-yl)-benzoic acid hydrochloride is obtained.

MS ESI (M+H)⁺=456.17

### Example 74

### Ethyl-3-(3-{4-[(4-Fluorobenzyl)-methylamino]-butyl}-ureido)-5-(1-methyl-1H-tetrazol-5-yl)-benzoate

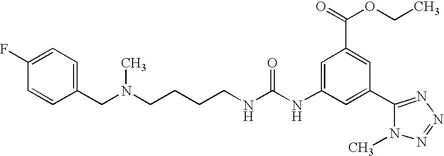

A slurry of 60 mg of 3-(3-{4-[(4-fluoro-benzyl)-methyl-amino]-butyl}-ureido)-5-(1-methyl-1H-tetrazol-5-yl)-benzoic acid hydrochloride in 1 ml of EtOH is treated with 0.0065 ml of concentrated sulfuric acid and heated in a microwave at 130° for 10 minutes. The mixture is concentrated and ethyl-3-(3-{4-[(4-Fluoro-benzyl)-methyl-amino]-butyl}-ureido)-5-(1-methyl-1H-tetrazol-5-yl)-benzoate is obtained.

MS APCI (M+H)⁺=484.24

### Example 75

### 1-{4-[(4-Fluorobenzyl)-methylamino]-butyl}-3-[3-hydroxymethyl-5-(1-methyl-1H-tetrazol-5-yl)-phenyl]-urea

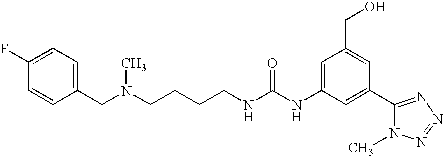

To a slurry of 5.00 g of 3-(1-methyl-1H-tetrazol-5-yl)-5-nitro-benzoic acid in 100 ml of anhydrous THF, 40 ml of 1M borane/THF complex are added at 0°. The mixture obtained is stirred at reflux temperature for 2 hours. After cooling, 100 ml of 1N HCl are added, and the mixture obtained is stirred at 80° for 30 minutes. After cooling and exhaustive extraction with EtOAc, the combined organic extracts obtained are dried, concentrated, and purified by silica gel chromatography. [3-(1-Methyl-1H-tetrazol-5-yl)-5-nitro-phenyl]-methanol is obtained.

Hydrogen gas is bubbled through a slurry of 115 mg of [3-(1-methyl-1H-tetrazol-5-yl)-5-nitrophenyl]-methanol and 10 mg of palladium on activated charcoal in 4 ml of MeOH for 6 hours at ambient temperature. The mixture obtained is filtered through celite and concentrated. [3-Amino-5-(1-methyl-1H-tetrazol-5-yl)-phenyl]-methanol and 3-methyl-5-(1-methyl-1H-tetrazol-5-yl)phenylamine are obtained after separation via silica gel chromatography.

To a mixture of 57 mg of [3-amino-5-(1-methyl-1H-tetrazol-5-yl)-phenyl]-methanol and 0.09 ml of pyridine in 2 ml of CH₂Cl₂, 0.071 ml of phenyl chloroformate at 0° are added. After stirring for 60 minutes at ambient temperature, the mixture obtained is quenched with saturated aq. sodium hydrogen carbonate solution. After exhaustive extraction with CH₂Cl₂, the combined organic extracts obtained are dried and concentrated.

[3-Hydroxymethyl-5-(1-methyl-1H-tetrazol-5-yl)-phenyl]-carbamic acid phenyl ester is obtained. A solution of 70 mg of intermediate E, 96 mg of [3-hydroxymethyl-5-(1-methyl-1H-tetrazol-5-yl)phenyl]-carbamic acid phenyl ester, and 0.103 ml of Et₃N in 2 ml of CH₂Cl₂ is stirred for 24 hours at ambient temperature. The mixture obtained is partitioned between dilute ammonium hydroxide and CH₂Cl₂. After exhaustive extraction with CH₂Cl₂ the combined organic extracts obtained are dried, concentrated, and purified by silica gel chromatography. The product obtained is treated with 17 mg of lithium hydroxide monohydrate in a 3:1:1 mixture of THF/H₂O/MeOH for 4 hours at ambient temperature. The mixture obtained is partitioned between dilute ammonium hydroxide and CH₂Cl₂. After exhaustive extraction with CH₂Cl₂ the combined organic extracts obtained are dried and concentrated.

1-{4-[(4-Fluoro-benzyl)-methyl-amino]-butyl}-3-[3-hydroxymethyl-5-(1-methyl-1H-tetrazol-5-yl)phenyl]-urea is obtained.

MS ESI (M+H)⁺=442.25

The following compound is prepared analogously as described in the previous example:

### Example 76

### 1-{4-[(4-Fluorobenzyl)-methylamino]-butyl}-3-[3-methyl-5-(1-methyl-1H-tetrazol-5-yl)-phenyl]-urea

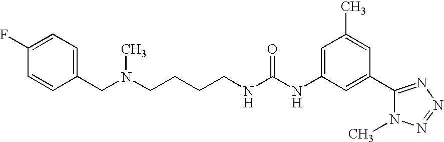

MS ESI (M+H)⁺=426.26

### Example 77

### 1-(5-Acetyl-4-methyl-thiazol-2-yl)-3-{4-[(4-fluorobenzyl)-methylamino]-butyl}-urea

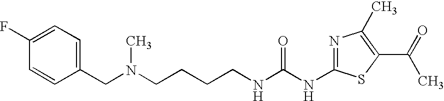

To an ice-cold solution of 1.00 g of 5-acetyl-2-amino-4-methylthiazole and 1.5 mL of pyridine, 1.2 ml of phenyl chloroformate are added. After stirring for 10 minutes at ambient temperature, the mixture obtained is quenched with saturated aq. sodium hydrogen carbonate solution. After exhaustive extraction with CH₂Cl₂, the combined organic extracts are dried and concentrated.

(5-Acetyl-4-methyl-thiazol-2-yl)-carbamic acid phenyl ester is obtained.

A solution of 200 mg of intermediate E, 193 mg of (5-acetyl-4-methyl-thiazol-2-yl)-carbamic acid phenyl ester, and 0.287 ml of Et₃N in 5 ml of CH₂Cl₂ is stirred for 24 hours at ambient temperature. The mixture obtained is partitioned between saturated aq. sodium hydrogen carbonate solution and CH₂Cl₂. After exhaustive extraction with CH₂Cl₂ the combined organic extracts obtained are dried and concentrated.

1-(5-Acetyl-4-methyl-thiazol-2-yl)-3-{4-[(4-fluoro-benzyl)-methyl-amino]-butyl}-urea is obtained.

MS APCI (M+H)⁺=393.19

The following compound is prepared analogously as described in the previous example:

### Example 78

### 1-(5-Acetyl-4-methyl-thiazol-2-yl)-3-{4-[(3,4-dimethylbenzyl)-methylamino]-butyl}-urea

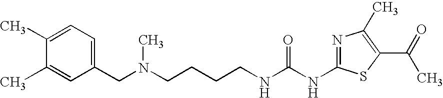

MS ESI (M+H)⁺=403.20

### Example 79

### 1-{4-[(4-Fluorobenzyl)-methylamino]-butyl}-3-[5-(1-methyl-1H-tetrazol-5-yl)pyridin-3-yl]-urea

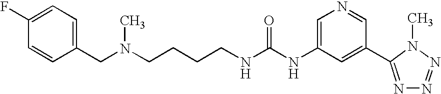

To a solution of 5.00 g of 5-bromonicotinic acid in 30 ml of DMF are added 1.99 g of methylamine hydrochloride, 3.31 g of 1-hydroxybenzotriazole, 9.54 g of 1-(3-dimethylaminopropyl)-3-ethylcarbodiimide hydrochloride, and 8.04 ml of N-methylmorpholine. After stirring for 24 hours at ambient temperature, the mixture obtained is quenched with saturated aq. sodium hydrogen carbonate solution. After exhaustive extraction with EtOAc, the combined organic extracts obtained are dried and concentrated. 5-Bromo-N-methyl-nicotinamide is obtained.

To an ice-cold solution of 1.1 g of 5-bromo-N-methyl-nicotinamide in 50 ml of anhydrous ACN, 0.67 g of sodium azide are added. After stirring for 30 minutes, 0.86 ml of trifluoromethanesulfonic anhydride are added, the mixture obtained is stirred at ambient temperature for 60 minutes.

Subsequently, 0.67 g of sodium azide are added. After 30 minutes, 2.58 ml of trifluoromethanesulfonic anhydride are added. The mixture obtained is stirred at ambient temperature for 24 hours and quenched with saturated aq. sodium hydrogen carbonate solution. After exhaustive extraction with EtOAc, the combined organic extracts obtained are dried and concentrated. 3-Bromo-5-(1-methyl-1H-tetrazol-5-yl)-pyridine is obtained.

A solution of 1.4 g of 3-bromo-5-(1-methyl-1H-tetrazol-5-yl)-pyridine and 288 mg of copper sulfate pentahydrate in 20 ml of 25% aq. ammonia is heated in a microwave at 150° for 4 hours. The mixture is concentrated. 5-(1-methyl-1H-tetrazol-5-yl)-pyridin-3-ylamine is obtained.

To a mixture of 400 mg of 5-(1-methyl-1H-tetrazol-5-yl)-pyridin-3-ylamine and 0.54 ml of pyridine in 3 ml of CH₂Cl₂, 0.572 ml of phenyl chloroformate are added at 0° C. After stirring for 10 minutes at ambient temperature, the mixture obtained is quenched with saturated aq. sodium hydrogen carbonate solution. After exhaustive extraction with EtOAc, the combined organic extracts obtained are dried and concentrated.

[5-(1-Methyl-1H-tetrazol-5-yl)-pyridin-3-yl]-carbamic acid phenyl ester is obtained.

A solution of 162 mg of N-1-(4-fluorobenzyl)-N-1-methylbutane-1,4-diamine. 2HCl, 169 mg of [5-(1-methyl-1H-tetrazol-5-yl)-pyridin-3-yl]-carbamic acid phenyl ester, and 0.235 ml of Et₃N in 3 ml of CH₂Cl₂ is stirred for 24 hours at ambient temperature. The mixture obtained is partitioned between saturated aq. sodium hydrogen carbonate solution and EtOAc. After exhaustive extraction with EtOAc the combined organic extracts obtained are dried and concentrated. 1-{4-[(4-Fluoro-benzyl)methyl-amino]-butyl}-3-[5-(1-methyl-1H-tetrazol-5-yl)-pyridin-3-yl]-urea is obtained.

MS ESI (M+H)⁺=413.18

### Example 80

### 2-(3-{4-[(4-Fluorobenzyl)-methylamino]-butyl}-ureido)-4-methyl-thiazole-5-carboxylic acid dimethylamide

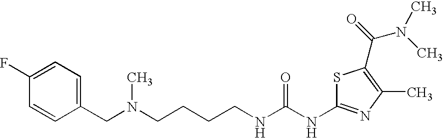

To a solution of 1.00 g of 2-amino-4-methyl-thiazole-5-carboxylic acid ethyl ester, 0.957 ml of Et₃N, and 1.73 g of di-f-butyl dicarbonate in 30 ml of THF, 65 mg of dimethylaminopyridine are added. After stirring for 2 hours at ambient temperature, the mixture obtained is adjusted to pH 6 by adding 1N HCl. After exhaustive extraction with EtOAc, the combined organic extracts obtained are dried and concentrated.

2-tert-Butoxycarbonylamino-4-methyl-thiazole-5-carboxylic acid ethyl ester is obtained.

1.52 g of 2-tert-butoxycarbonylamino-4-methyl-thiazole-5-carboxylic acid ethyl ester are dissolved in a 3:1:1 mixture of THF/H₂O/MeOH. 1.09 g of lithium hydroxide monohydrate are added, and the mixture obtained is stirred for 24 hours at 50°. After cooling and addition of 1N HCl, the mixture obtained is exhaustively extracted with EtOAc. The combined organic extracts obtained are dried and concentrated.

2-tert-Butoxycarbonylamino-4-methyl-thiazole-5-carboxylic acid is obtained.

To a solution of 1.00 g of 2-tert-butoxycarbonylamino-4-methyl-thiazole-5-carboxylic acid in 30 ml of DMF, 375 mg of methylamine hydrochloride, 518 mg of 1-hydroxy benzotriazole, 1.49 g of 1-(3-dimethylaminopropyl)-3-ethylcarbodiimide hydrochloride and 2.61 mL of N-ethyldiisopropylamine are added. After stirring for 24 hours at ambient temperature, the mixture obtained is quenched with saturated aq. sodium hydrogen carbonate solution. After exhaustive extraction with EtOAc, the combined organic extracts obtained are dried and concentrated.

(5-Dimethylcarbamoyl-4-methyl-thiazol-2-yl)-carbamic acid tert-butyl ester is obtained. 800 mg of (5-dimethylcarbamoyl-4-methyl-thiazol-2-yl)-carbamic acid tert-butyl ester are treated with 15 ml of 4N HCl in dioxane for 24 hours at ambient temperature. The mixture is concentrated and 2-amino-4-methyl-thiazole-5-carboxylic acid dimethylamide hydrochloride salt is obtained.

To a mixture of 609 mg of 2-amino-4-methyl-thiazole-5-carboxylic acid dimethylamide hydrochloride salt and 0.65 ml of pyridine in 10 ml of CH₂Cl₂, 0.691 ml of phenyl chloroformate are added at 0°. After stirring for 10 minutes at ambient temperature, the mixture obtained is quenched with saturated aq. sodium hydrogen carbonate solution. After exhaustive extraction with EtOAc, the combined organic extracts obtained are dried and concentrated.

(5-Dimethylcarbamoyl-4-methyl-thiazol-2-yl)-carbamic acid phenyl ester is obtained.

A solution of 200 mg of N-1-(4-fluorobenzyl)-N-1-methylbutane-1,4-diamine. 2HCl, 216 mg of (5-dimethylcarbamoyl-4-methyl-thiazol-2-yl)-carbamic acid phenyl ester and 0.290 ml of Et₃N in 3 ml of CH₂Cl₂ is stirred for 24 hours at ambient temperature. The mixture obtained is partitioned between saturated aq. sodium hydrogen carbonate solution and EtOAc. After exhaustive extraction with EtOAc the combined organic extracts obtained are dried and concentrated. 2-(3-{4-[(4-Fluorobenzyl)-methyl-amino]-butyl}-ureido)-4-methyl-thiazole-5-carboxylic acid dimethylamide is obtained.

MS ESI (M+H)⁺=422.18

The following compound is prepared analogously as described in the previous example:

### Example 81

### 2-(3-{4-[(3,4-Dimethylbenzyl)-methylamino]-butyl}-ureido)-4-methyl-thiazole-5-carboxylic acid dimethylamide

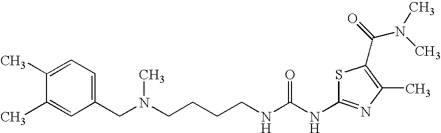

MS ESI (M+H)⁺=432.24

### Example 82

### 1-[3-(4,5-Dimethyl-1H-imidazol-2-yl)-phenyl]-3-{4-[(4-fluorobenzyl)methylamino]-butyl}-urea

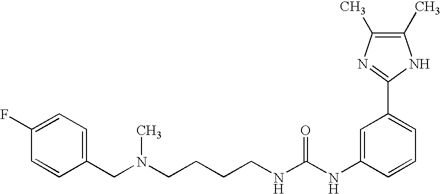

1 g of 3-Nitrobenzaldehyde, 419 μl of 2,3-butanedione, 3.61 g of ammonium acetate and 5 ml of acetic acid are heated in a microwave for 5 minutes at 120°. The mixture obtained is diluted with EtOAc, washed with saturated aq. sodium hydrogen carbonate solution, dried and concentrated.

4,5-Dimethyl-2-(3-nitro-phenyl)-1H-imidazole is obtained.

910 mg of 4,5-Dimethyl-2-(3-nitro-phenyl)-1H-imidazole in 60 ml of ACN are treated with 1.13 g of di-tert-butyl dicarbonate and 665 mg of 4-dimethylaminopyridine at 0°. The solution obtained is stirred at ambient temperature for 18 hours. The mixture obtained is diluted with EtOAc, washed with saturated aq. ammonium chloride solution, dried and concentrated.

4,5-Dimethyl-2-(3-nitro-phenyl)-imidazole-1-carboxylic acid tert-butyl ester is obtained.

1.12 g of 4,5-Dimethyl-2-(3-nitro-phenyl)-imidazole-1-carboxylic acid tert- and stirred under an atmosphere of hydrogen for 4 hours. The mixture obtained is filtered through celite and the filtrate obtained is concentrated.

2-(3-Amino-phenyl)-4,5-dimethyl-imidazole-1-carboxylic acid tert-butyl ester is obtained.

18 mg of Triphosgene are dissolved in 5 ml of CH₂Cl₂ and cooled to −78°. A solution of 51 mg of 2-(3-amino-phenyl)-4,5-dimethyl-imidazole-1-carboxylic acid tert-butyl ester and 74 μl of triethylamine in 2 ml of CH₂Cl₂ is added slowly. The mixture obtained is stirred for 1 hour, warmed to ambient temperature and concentrated. The residue obtained is dissolved in 7 ml of CH₂Cl₂ and a solution of 50 mg of N-1-(4-fluorobenzyl)-N-1-methylbutane-1,4-diamine. 2HCl and 150 μl of triethylamine in 2 ml of CH₂Cl₂ is added slowly at ambient temperature. The solution obtained is stirred for 18 hours. The mixture is diluted with CH₂Cl₂, washed with brine, dried and concentrated.

2-[3-(3-{4-[(4-Fluoro-benzyl)-methyl-amino]-butyl}-ureido)-phenyl]-4,5-dimethyl-imidazole-1-carboxylic acid tert-butyl ester is obtained.

25 mg of 2-[3-(3-{4-[(4-Fluoro-benzyl)-methyl-amino]-butyl}-ureido)-phenyl]-4,5-dimethyl-imidazole-1-carboxylic acid tert-butyl ester in 5 ml of MeOH are treated with 5 ml of hydrochloric acid (5M in H₂O) at ambient temperature for 30 minutes. The mixture obtained is concentrated.

1-[3-(4,5-Dimethyl-1H-imidazol-2-yl)-phenyl]-3-{4-[(4-fluoro-benzyl)-methyl-amino]-butyl}-urea is obtained.

MS ESI (M+H)⁺=424.30

The following compound is prepared analogously to example 82:

### Example 83

### 1-[3-(4,5-Dimethyl-1H-imidazol-2-yl)-5-(1-methyl-1H-tetrazol-5-yl)-phenyl]-3-{4-[(4-fluoro-benzyl)-methylamino]-butyl}-urea

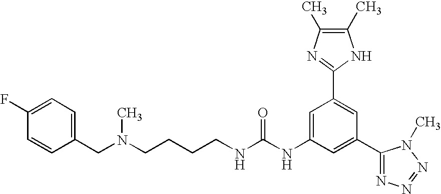

MS APCI (M−H)⁺=504.31

### Example 84

### 1-{4-[(4-Fluorobenzyl)-methyl-amino]-butyl}-3-[3-(5-methyl-[1,3,4]oxadiazol-2-yl)-phenyl]-urea

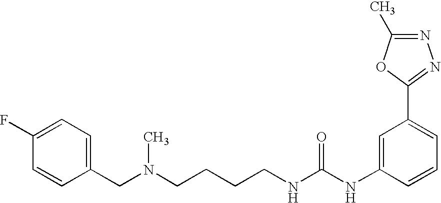

2.40 g of Acetylhydrazide in 5 ml of THF are added slowly to a solution of 600 mg of 3-nitrobenzoylchloride in 15 ml of THF at 0°. The solution obtained is allowed to stir for 2 hours. The mixture obtained is concentrated and 3-nitrobenzoic acid N′-acetyl-hydrazide is obtained.

280 mg of 3-Nitrobenzoic acid N′-acetyl-hydrazide and 616 mg of Burgess' reagent are heated in 6 ml of THF in a microwave for 5 minutes at 100°. The mixture obtained is concentrated.

2-Methyl-5-(3-nitro-phenyl)-[1,3,4]oxadiazole is obtained.

340 mg of 2-Methyl-5-(3-nitro-phenyl)-[1,3,4]oxadiazole and 5 mg of palladium on charcoal (10%) in 30 ml of EtOAc are flushed with hydrogen gas and stirred under an atmosphere of hydrogen for 4 hours. The mixture obtained is filtered through celite and the filtrate obtained is concentrated. 3-(5-Methyl-[1,3,4]oxadiazol-2-yl)-phenylamine is obtained.

21 mg of Triphosgene are dissolved in 5 ml of CH₂Cl₂ and cooled to −78°. A solution of 31 mg of methyl-[1,3,4]oxadiazol-2-yl)-phenylamine and 86 μl of triethylamine in 2 ml of CH₂Cl₂ are added slowly. The mixture obtained is stirred for 3 hours, warmed to ambient temperature and concentrated. The residue obtained is dissolved in 7 ml of CH₂Cl₂ and a solution of 50 mg of N-1-(4-fluorobenzyl)-N-1-methylbutane-1,4-diamine. 2HCl and 172 μl of triethylamine in 2 ml of CH₂Cl₂ are added slowly at ambient temperature. The solution obtained is stirred for 18 hours. The mixture obtained is diluted with CH₂Cl₂, washed with brine, dried and concentrated. 1-{4-[(4-Fluoro-benzyl)methyl-amino]-butyl}-3-[3-(5-methyl-[1,3,4]oxadiazol-2-yl)-phenyl]-urea is obtained.

MS ES (M+H)⁺=412.25

The following compound is prepared analogously to example 84:

### Example 85

### 1-{4-[(4-Fluorobenzyl)-methylamino]-butyl}-3-[3-(5-methyl-[1,3,4]oxadiazol-2-yl)-5-(1-methyl-1H-tetrazol-5-yl)-phenyl]-urea

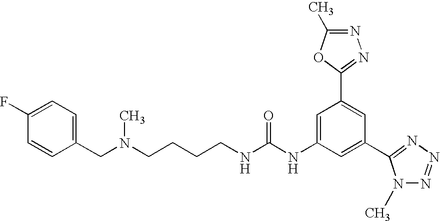

MS ES (M+H)⁺=494.28

### Preparation of Intermediates:

### A) [3-(1-Methyl-1H-tetrazol-5-yl)-phenyl]-carbamic acid phenyl ester

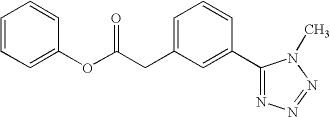

41 ml of methyl amine in THF are added to 7.05 g of 3-nitrobenzoyl chloride in 150 ml of THF and stirred at ambient temperature for 2 hours. The reaction mixture obtained is diluted with EtOAc and washed 3 times with H₂O, brine, dried, filtered and concentrated. N-methyl-3-nitrobenzamide is obtained.

6.62 g of N-methyl-3-nitrobenzamide in 200 ml of ACN are added to 2.39 g of sodium azide and the reaction mixture obtained is cooled to 0°. 10 g of trifluoromethanesulfonic anhydride are added drop-wise and the solution obtained is stirred at this temperature for 3.5 hours. The reaction mixture obtained is diluted with CH₂Cl₂ and poured into 1M NaOH. The organic layer obtained is separated, washed with H₂O, brine, dried, filtered and concentrated.

5-(3-nitrophenyl)-1-methyl-1H-tetrazole is obtained.

To 2.0 g of 5-(3-nitrophenyl)-1-methyl-1H-tetrazole in an EtOAc (25 ml) and MeOH (75 ml) solution, Pd/C (10%) is added under N₂. The reaction mixture obtained is flushed with N₂ then with H₂. The hydrogenation reaction is conducted at 55 psi for 1 hour, the reaction mixture obtained is filtered through a plug of Celite and the filtrate obtained is concentrated. 3-(1-methyl-1H-tetrazol-5-yl) is obtained.

0.39 ml of phenyl chloroformate are added drop-wise to 539 mg of 3-(1-methyl-1H-tetrazol-5-yl)aniline) and 0.81 ml of 2,6-lutidine in 10 ml of DCM and the reaction mixture obtained is stirred for 2 hours at ambient temperature. The reaction mixture obtained is poured into 1.0 M HCl and extracted with CH₂Cl₂. The combined organic layers obtained are washed with H₂O, brine, dried, filtered and concentrated.

Phenyl 3-(1-methyl-1H-tetrazol-5-yl)phenylcarbamate is obtained

The following intermediates are prepared analogously to intermediate A:

### B) [3,5-Bis-(1-methyl-1H-tetrazol-5-yl)-phenyl]-carbamic acid phenyl ester

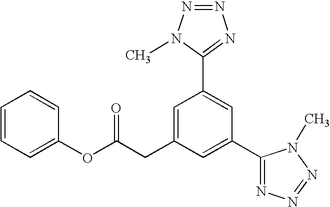

### C) Preparation of 3-bromo-5-nitrophenyl)-1-methyl-1H-tetrazole

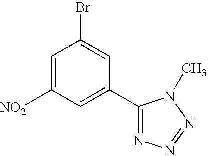

26.7 g of N-bromosuccinimide are added portion-wise to a stirred solution of 16.7 g of 3-nitrobenzoic acid in 50 ml of trifluoroacetic acid and 20 ml of sulfuric acid at 50°. The mixture obtained is stirred for 16 hours, cooled, poured into 200 ml of ice H₂O and extracted 3 times with 100 ml of EtOAc. The combined organic layers obtained are dried, filtered, and concentrated, then triturated with DCM. 3-bromo-5-nitrobenzoic acid is obtained.

1 ml of N,N-dimethylformamide is added to a solution of 17.7 g of 3-bromo-5-nitrobenzoic acid and 12.6 ml of oxalyl chloride in 250 ml of DCM and the mixture obtained is stirred at ambient temperature for 5 hours. The reaction mixture obtained is concentrated and re-concentrated twice with 100 ml of toluene to remove excess of oxalyl chloride. The residue obtained is dissolved in 100 ml of THF and added drop-wise to a solution of 108 ml of methylamine in THF at 0°. The mixture obtained is stirred at ambient temperature for 14 hours, filtered and the filtrate obtained is concentrated. 3-bromo-N-methyl-5-nitrobenzamide is obtained.

1.15 ml of sodium azide are added to a suspension of 4.6 g of 3-bromo-N-methyl-5-nitrobenzamide in 100 ml of ACN at ambient temperature. The reaction mixture obtained is cooled to 0° prior to cautious addition of 5.5 g of triflic anhydride and stirred for 4 hours at this temperature. The solution obtained is quenched by addition of saturated aq. sodium hydrogen carbonate solution and extracted 3 times with 20 ml of EtOAc. The combined organic layers obtained are washed with sat. NaHCO₃, brine, dried, filtered and concentrated. 3-bromo-5-nitrophenyl)-1-methyl-1H-tetrazole is obtained.

### D) (3,5-bis-dimethylcarbamoylphenyl)carbamic acid phenyl ester

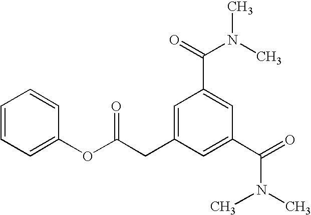

A mixture of 500 mg 5-nitroisophthalic acid and 5 ml SOCl₂ is stirred at 70° C. for 24 hours. After thorough evaporation of excess reagent, 10 ml of a 2M solution of dimethylamine in THF is added and the mixture is stirred at ambient temperature for 4 hours. After adding saturated aq. sodium hydrogen carbonate solution and exhaustive extraction with EtOAc, the combined organic extracts are dried (Na₂SO₄) and concentrated.

N,N,N′,N′-tetramethyl-5-nitro-isophthalamide is obtained.

600 mg N,N,N′,N′-tetramethyl-5-nitro-isophthalamide is dissolved in 15 ml MeOH and a catalytic amount of 10% palladium on activated charcoal is added. The mixture is hydrogenated at ambient temperature and atmospheric pressure for 2 hours, filtered through celite, and concentrated.

5-amino-N,N,N′,N′-tetramethylisophthalamide is obtained.

533 mg 5-amino-N,N,N′,N′-tetramethylisophthalamide is dissolved in DCM and 0.54 g pyridine and 0.53 mg phenyl chloroformate are added. After stirring at ambient temperature for 60 min, the mixture is concentrated.

(3,5-bis-dimethylcarbamoylphenyl)carbamic acid phenyl ester is obtained.

### E) Preparation of N-1-(4-fluorobenzyl)-N-1-methylbutane-1,4-diamine.2HCl

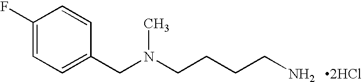

N-(4-bromobutyl)phthalimide (1.1 equiv) and potassium iodide (catalytic amount) are added to a solution of 4-fluoro N-methylbenzylamine (1 equiv) and Et₃N (1.1 equiv) in ACN at ambient temperature. The reaction mixture obtained is stirred at this temperature for 18 hours, filtered and concentrated.

N′—(N-4-(4-fluorobenzyl)-N-4-methylbutylamino)phthalimide is obtained.

Hydrazine monohydrate (1.3 equivalents) is added to a solution of N′—(N-4-(4-fluoro-benzyl)-N-4-methylbutylamino)phthalimide (1.0 equiv) in EtOH and the mixture obtained is heated at reflux for 2 hours. To the mixture obtained, concentrated HCl is added drop-wise (pH 3-4) and the reaction mixture obtained is heated until complete by TLC (2-6 h). On cooling, the reaction mixture obtained is concentrated and filtered. The solid obtained is washed 3 times with ice cold MeOH, the filtrate obtained is concentrated and triturated with diethyl ether.

N-1-(4-fluorobenzyl)-N-1-methylbutane-1,4-diamine.2HCl is obtained.

The following intermediate is prepared analogously to intermediate E:

### F) N*1*-(3,4-Dimethyl-benzyl)-N*1*-methyl-butane-1,4-diamine dihydrochloride

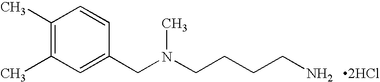

### G) N-cyclopropyl-N-(4-fluorobenzyl)butane-1,4-diamine

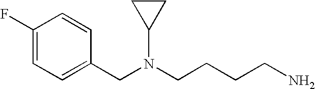

15.3 g Sodium triacetoxyborohydride is added to a stirred solution of 5 ml 4-fluorobenzaldehyde and 6.57 ml cyclopropylamine in 70 ml THF. The mixture is stirred at ambient temperature for 24 hours. After quenching with saturated aq. sodium hydrogen carbonate solution and exhaustive extraction with EtOAc, the combined organic extracts are dried and concentrated.

Cyclopropyl-(4-fluorobenzyl)amine is obtained.

A mixture of 1.0 g cyclopropyl-(4-fluorobenzyl)amine, 1.67 g N-(4-bromobutyl)phthalimide, 1.85 ml triethylamine and 9 mg potassium iodide in 20 ml MeCN is stirred at 80° C. for 4 hours. After evaporation of the solvent, the residue is partitioned between saturated aq. sodium hydrogen carbonate solution and EtOAc. After exhaustive extraction with EtOAc, the combined organic extracts are dried (Na₂SO₄) and concentrated.

2-{4-[cyclopropyl-(4-fluorobenzyl)amino]butyl}isoindole-1,3-dione is obtained.

A solution of 765 mg 2-{4-[cyclopropyl-(4-fluorobenzyl)amino]butyl}isoindole-1,3-dione in 40 ml MeOH is treated with 1.05 g hydrazine hydrate at ambient temperature for 2 hours. After evaporation of the solvent, the residue is triturated with EtOAc. The insoluble material is separated by filtration, and the filtrate is concentrated.

N-cyclopropyl-N-(4-fluorobenzyl)butane-1,4-diamine is obtained.

### H) Preparation of (E)-N-(4-fluorobenzyl)-N-methyl-but-2-ene-1,4-diamine

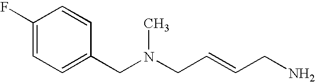

A mixture of trans-1,4-dibromo-2-butene (1.0 equiv) and potassium phthalimide (0.5 equiv) in DMF is heated at 125° in an inert atmosphere for 3 hours and solvent is evaporated.

2-((E)-4-bromo-but-2-enyl)-isoindole-1,3-dione is obtained.

Potassium carbonate (1.1 equiv) is added to a mixture of 2-((E)-4-bromo-but-2-enyl)-isoindole-1,3-dione (1.0 equiv) and N-(4-fluorobenzyl)-N-methylamine (1.0 equiv) in DMF in an inert atmosphere and heated at 60° for 2 hours. After cooling, the mixture obtained is partitioned between H₂O and EtOAc, the aq. layer obtained is extracted 3 times with EtOAc. The combined organic layers obtained are dried, filtered and concentrated.

2-{(E)-4-[(4-fluoro-benzyl)-methylamino]-but-2-enyl}-isoindole-1,3-dione is obtained.

A solution of 2-{(E)-4-[(4-fluoro-benzyl)-methylamino]-but-2-enyl}-isoindole-1,3-dione (1.0 equiv) in MeOH is treated with hydrazine hydrate (10.0 equiv) at ambient temperature. After stirring for 60 minutes, solvent is evaporated, the mixture obtained is triturated with EtOAc and the insoluble material is filtered off. Solvent from the filtrate obtained is evaporated.

(E)-N-(4-fluorobenzyl)-N-methyl-but-2-ene-1,4-diamine is obtained.

### I) Preparation of (±)-4-amino-1-[(4-fluorobenzyl)methylamino]butan-2-ol bis-trifluoroacetate

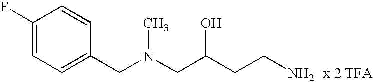

Di-t-butyl dicarbonate (1 equiv) and triethylamine (2.2 equiv) are added subsequently to 3-butene-1-amine hydrochloride (1 equiv) in CH₂Cl₂. The mixture obtained is stirred in an inert atmosphere for 24 hours at ambient temperature and quenched with an saturated aq. ammonium chloride solution. After extraction with CH₂Cl₂, the combined organic layers obtained are dried, filtered and concentrated.

t-Butyl but-3-enyl-carbamate is obtained.

3-chloro-perbenzoic acid (3 equiv) is added to an ice-cooled solution of t-butyl but-3-enyl-carbamate (1 equiv) in CH₂Cl₂ in an inert atmosphere and stirred at 40° for 3 hours. The mixture obtained is cooled to 5° and quenched with saturated aq. sodium bisulfite solution. After extraction with CH₂Cl₂, the combined organic layers obtained are washed 3 times with saturated aq. potassium carbonate solution, dried, filtered and concentrated.

(±)-t-butyl (2-oxiranyl-ethyl)-carbamate is obtained.

4-Fluoro-N-methyl-benzylamine (1.0 equiv) and (±)-t-butyl (2-oxiranyl-ethyl)-carbamate (1.0 equiv) are heated in anhydrous EtOH in an inert atmosphere at 60° for 3 hours. The mixture obtained is cooled and quenched with saturated aq. sodium carbonate solution. After extraction with EtOAc, the combined organic layers obtained are dried, filtered and concentrated.

(±)-t-butyl {4-[(4-fluoro-benzyl)-methyl-amino]-3-hydroxy-butyl}-carbamate is obtained.

(±)-t-Butyl {4-[(4-fluoro-benzyl)-methyl-amino]-3-hydroxy-butyl}-carbamate (1.0 equiv) in CH₂Cl₂ are treated with trifluoroacetic acid (30 equiv) in an inert atmosphere at ambient temperature. After 2 hours, the mixture obtained is concentrated and dried.

(±)-4-Amino-1-[(4-fluorobenzyl)-methyl-amino]-butan-2-ol bis-trifluoroacetate is obtained.

### J) Preparation of 4-fluoro-3-hydroxy-benzaldehyde

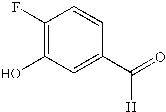

A solution of 1.00 g of 4-fluoro-3-methoxybenzaldehyde in 25 ml of CH₂Cl₂ is cooled to −78°. 19 ml of boron tribromide (1M in CH₂Cl₂) are added slowly, and the mixture obtained is allowed to reach ambient temperature and is stirred for 18 hours. The mixture obtained is poured on ice-H₂O and stirred for 60 minutes. The organic layer obtained is separated, washed with saturated aq. sodium hydrogen carbonate solution and extracted twice with 2N NaOH. The combined alkaline extracts obtained are acidified with concentrated HCl and extracted exhaustively with CH₂Cl₂. The combined organic extracts obtained are washed with brine, dried and concentrated.

4-Fluoro-3-hydroxy-benzaldehyde is obtained.

### K) Preparation of 3-cyclopropylmethoxy-4-fluoro-benzaldehyde

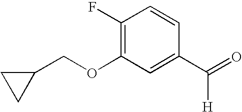

To a solution of 261 mg of 4-fluoro-3-hydroxy-benzaldehyde in 4 ml of DMF are added 286 mg of potassium carbonate and 0.356 ml of (bromomethyl)cyclopropane. The mixture obtained is stirred at 100° for 4 hours. After cooling, the mixture obtained is diluted with H₂O and extracted exhaustively with CH₂Cl₂. The combined organic extracts obtained are washed with brine, dried and concentrated. 3-Cyclopropylmethoxy-4-fluoro-benzaldehyde is obtained.

1. A compound of formula 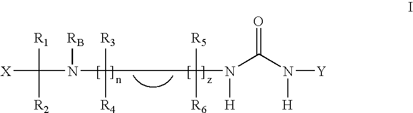
wherein
X is (C₆₋₁₈)aryl substituted one or more times by halogen, cyano, hydroxy, carboxy, nitro,
(C₁₋₈)alkyl, (C₁₋₈)alkoxy, (C₃₋₈)cycloalkyl, (C₃₋₈)cycloalkyl(C₁₋₄)alkoxy or (C₁₋₈)alkylamine, or unsubstituted or substituted (C₆₋₁₈)aryl annelated with (C₃₋₈)cycloalkyl,
R₁, R₂, R₃, R₄, R₅ and R₆ are independently selected from the group consisting of hydrogen, cyano, hydroxy, carboxy, nitro, (C₁₋₈)alkyl and (C₁₋₈)alkoxy,
Rₐ is (C₁₋₄)alkyl, (C₃₋₈)cycloalkyl,
n is zero or an integer from 1 to 8, [symbol]
is a single bond, a double bond, (C₃₋₈)cycloalkyl or absent,
z is an integer from 1 to 5,
Y is a cyclic group selected from the group consisting of (C₆₋₁₈)aryl and an optionally
annelated 5 to 6 membered heterocyclic ring system, wherein at least one of the ring atoms is selected from N, O or S, said cyclic group being substituted one or more times by
halogen, (C₁₋₈)alkyl, (C₃₋₈)cycloalkyl, hydroxyl(C₁₋₄)alkyl, (C₁₋₈)alkoxycarbonyl, hydroxycarbonyl, (C₁₋₄)alkylcarbonylamino, (C₁₋₄)alkylaminocarbonyl, di(C₁₋₄)alkylaminocarbonyl, (C₆₋₁₈)arylaminocarbonyl, (C₆₋₁₈)aryl(C₁₋₂)alkylaminocarbonyl, (C₁₋₄)alkylamino(C₁₋₄)alkylaminocarbonyl, di(C₁₋₄)alkylamino(C₁₋₄)alkylaminocarbonyl, (C₃₋₆)cycloalkyl(C₁₋₄)alkylaminocarbonyl, (C₆₋₁₈)arylaminocarbonyl, (C₆₋₁₈)aryl(C₁₋₄)alkylaminocarbonyl,
heterocyclyl(C₁₋₄)alkyl or heterocyclylcarbonyl, wherein heterocyclyl is a 5 to 6 membered heterocyclic ring system, wherein at least one of the ring atoms is selected from N, O or S,
(C₆₋₁₈)aryl, or by an unsubstituted or substituted optionally annelated 5 to 6 membered heterocyclic ring system, wherein at least one of the ring atoms is selected from N, O or S.

2. A compound of formula I of claim 1, wherein
X is phenyl substituted one or more times by halogen, cyano, hydroxy, carboxy, nitro,
(C₁₋₄)alkyl, (C₁₋₄)alkoxy, (C₁₋₄)alkoxy, (C₃₋₆)cycloalkyl(C₁₋₂)alkoxy or (C₁₋₄)alkylamine, or
unsubstituted or substituted phenyl annelated with (C₃₋₈)cycloalkyl,
R₁, R₂, R₃, R₄, R₅ and R₆ are independently selected from the group consisting of hydrogen, cyano, hydroxy, carboxy, nitro, (C₁₋₈)alkyl and (C₁₋₈)alkoxy,
Rₐ is (C₁₋₂)alkyl or (C₃₋₆)cycloalkyl,
n is zero or an integer from 1 to 4, [symbol]
is a double bond, (C₃₋₈)cycloalkyl or absent,
z is an integer from 1 to 4,
Y is phenyl substituted one or more times by halogen, (C₁₋₈)alkyl, (C₃₋₆) cycloalkyl,
hydroxy(C₁₋₄)alkyl, (C₁₋₆)alkoxycarbonyl, hydroxycarbonyl, (C₁₋₄)alkylcarbonylamino, (C₁₋₄)alkylaminocarbonyl, di(C₁₋₄)alkylaminocarbonyl, (C₆₋₁₈)arylaminocarbonyl, (C₆₋₁₈)aryl(C₁₋₂)alkylaminocarbonyl, (C₁₋₄)alkylamino(C₁₋₄)alkylaminocarbonyl, di(C₁₋₄)alkylamino(C₁₋₄)alkylaminocarbonyl, (C₃₋₈)cycloalkyl(C₁₋₄)alkylaminocarbonyl, (C₆₋₁₈)arylaminocarbonyl, (C₆₋₁₈)aryl(C₁₋₄)alkylaminocarbonyl,
heterocyclyl(C₁₋₂)alkyl or heterocyclylcarbonyl, wherein heterocyclyl is a 5 to 6 membered heterocyclic ring system, wherein at least one of the ring atoms is selected from N, O or S, or by unsubstituted or substituted
phenyl, tetrazolyl, tetrazolyl-methyl, benzothiazolyl, thiophenyl, pyrazolyl, furanyl, 2,3-dihydro-1H-indolyl, pyridinyl, 1,3,4-oxazolyl, 1H-imidazolyl, thiazolyl, or
unsubstituted or substituted thiazolyl or pyridinyl.

3. A compound of formula I of claim 1, wherein
X is phenyl one to threefold substituted by fluoro, methyl, methoxy, hydroxy, cyclopropylmethoxy, or
phenyl annelated with cyclopentyl,
R₁, R₂, R₃, R₄, R₅ and R₆ are independently selected from the group consisting of hydrogen, and hydroxy,
Rₐ is methyl or cyclopropyl,
n is zero or 1, [symbol]
is a double bond, cyclopropyl or absent,
z is an integer from 1 to 4,
Y is phenyl one or twofold substituted by bromo, chloro, methyl, ethyl, hydroxymethyl, hydroxycarbonyl, ethoxycarbonyl, t-butoxycarbonyl, methylcarbonylamino,
methylaminocarbonyl, ethylaminocarbonyl, i-propylaminocarbonyl, dimethylaminocarbonyl,
dimethylaminoethylaminocarbonyl, cyclopropylmethylaminocarbonylamino, phenylaminocarbonyl, benzylaminocarbonyl, pyridin-2-yl-methylaminocarbonyl, unsubstituted tetrazolyl, 2H-tetrazol-5-yl-methyl, 1-methyl-1H-tetrazol-yl, 1-methyl-5H-tetrazol-1-yl, benzothiazol-2-yl substituted by 1-methyl-1H-tetrazol-1-yl, thiophen-2-yl, 1-benzyl-1H-pyrazol-4-yl, furan-2-yl, 2,3-dihydro-1H-indol-5-yl, pyridin-2-yl, pyridin-3-yl, pyridin-4-yl, tetrazol-5-yl-methyl, piperidine-1-carbonyl, morpholine-4-carbonyl, 4-methyl-piperazine-1-carbonyl, pyrrolidine-1-carbonyl, 1,3,4-oxadiazol-2-yl substituted by methyl, 1H-imidazol-2-yl one or twofold substituted by methyl,
thiazol-2-yl substituted by methyl, methylaminocarbonyl or dimethylaminocarbonyl, pyridin-3-yl substituted by 2-methyl-tetrazol-1-yl,
unsubstituted phenyl or phenyl substituted by methylaminocarbonyl,
thiazol-2-yl one or twofold substituted by methyl, methylcarbonyl, or dimethylaminocarbonyl or
pyridin-3-yl substituted by methyl-tetrazolyl.

4. A compound of claim 1 in the form of a salt.

5-6. (canceled)

7. A pharmaceutical composition comprising a compound of claim 1 in association with at least one pharmaceutical excipient.

8. A pharmaceutical composition according to claim 7, further comprising another pharmaceutically active agent.

9. A method of treatment of a disease mediated by CCR-3, which treatment comprises administering to a subject in need of such treatment an effective amount of a compound of formula (I) of claim 1.

10. A method of treatment of claim 9 wherein the disease is an inflammatory or allergic disease, particularly an inflammatory or obstructive airways disease.

11. A method of claim 9, wherein a compound of formula (I) according to claim 1 is administered in combination with another pharmaceutically active agent, either simultaneously or in sequence.

12. The compound of formula I of claim 2, wherein
X is phenyl one to threefold substituted by fluoro, methyl, methoxy, hydroxy, cyclopropylmethoxy, or
phenyl annelated with cyclopentyl,
R₁, R₂, R₃, R₄, R₅ and R₆ are independently selected from the group consisting of hydrogen, and hydroxy,
Rₐ is methyl or cyclopropyl,
n is zero or 1, [symbol]
is a double bond, cyclopropyl or absent,
z is an integer from 1 to 4,
Y is phenyl one or twofold substituted by bromo, chloro, methyl, ethyl, hydroxymethyl, hydroxycarbonyl, ethoxycarbonyl, t-butoxycarbonyl, methylcarbonylamino,
methylaminocarbonyl, ethylaminocarbonyl, i-propylaminocarbonyl, dimethylaminocarbonyl,
dimethylaminoethylaminocarbonyl, cyclopropylmethylaminocarbonylamino, phenylaminocarbonyl, benzylaminocarbonyl, pyridin-2-yl-methylaminocarbonyl, unsubstituted tetrazolyl, 2H-tetrazol-5-yl-methyl, 1-methyl-1H-tetrazol-1-yl, 1-methyl-5H-tetrazol-1-yl, benzothiazol-2-yl substituted by 1-methyl-1H-tetrazol-1-yl, thiophen-2-yl, 1-benzyl-1H-pyrazol-4-yl, furan-2-yl, 2,3-dihydro-1H-indol-5-yl, pyridin-2-yl, pyridin-3-yl, pyridin-4-yl, tetrazol-5-yl-methyl, piperidine-1-carbonyl, morpholine-4-carbonyl, 4-methyl-piperazine-1-carbonyl, pyrrolidine-1-carbonyl, 1,3,4-oxadiazol-2-yl substituted by methyl, 1H-imidazol-2-yl one or twofold substituted by methyl,
thiazol-2-yl substituted by methyl, methylaminocarbonyl or dimethylaminocarbonyl, pyridin-3-yl substituted by 2-methyl-tetrazol-1-yl,
unsubstituted phenyl or phenyl substituted by methylaminocarbonyl,
thiazol-2-yl one or twofold substituted by methyl, methylcarbonyl, or dimethylaminocarbonyl or
pyridin-3-yl substituted by methyl-tetrazolyl.

13. The compound of claim 2 in the form of a salt.

14. A pharmaceutical composition comprising a compound of claim 2 in association with at least one pharmaceutical excipient.

15. The method of claim 9, wherein the treatment comprises administering to a subject in need of such treatment an effective amount of a compound of formula (I) of claim 2.

16. The method of treatment of claim 15 wherein the disease is an inflammatory or allergic disease, particularly an inflammatory or obstructive airways disease.

17. The method of claim 9, wherein the treatment comprises administering to a subject in need of such treatment an effective amount of a compound of formula (I) of claim 3.

18. The method of treatment of claim 17 wherein the disease is an inflammatory or allergic disease, particularly an inflammatory or obstructive airways disease.

19. The method of claim 8, wherein the treatment comprises administering to a subject in need of such treatment an effective amount of the salt of claim 4.

20. The method of treatment of claim 19, wherein the disease is an inflammatory or allergic disease, particularly an inflammatory or obstructive airways disease.
